(19) United States
(12) Patent Application Publication
(10) Pub. No.: US 2022/0257803 A1
(43) Pub. Date: Aug. 18, 2022

(54) SUPERPARAMAGNETIC GOLD NANOPARTICLE CLUSTER-PROTEIN NANOPARTICLE FUSION BODY FOR MAGNETIC RESONANCE IMAGING AND MAGNETIC THERMOTHERAPY

(71) Applicant: CELLEMEDY INC., Seongnam-si (KR)

(72) Inventors: Jeewon LEE, Seoul (KR); Koo Chul KWON, Seoul (KR); Eunji JO, Seoul (KR)

(73) Assignee: CELLEMEDY INC., Seongnam-si (KR)

(21) Appl. No.: 17/722,727

(22) Filed: Apr. 18, 2022

Related U.S. Application Data
(63) Continuation-in-part of application No. 16/628,773, filed on Jan. 6, 2020.

(30) Foreign Application Priority Data
Jul. 4, 2017 (KR) 10-2017-0084793

(51) Int. Cl. A61K 49/18; A61K 41/00; A61K 47/69; A61P 35/00; C12N 15/86

(52) U.S. Cl. CPC A61K 49/1887; A61K 41/0052; A61K 47/6931; C12N 2730/10142; C12N 15/86; C12N 2730/10122; C12N 2730/10123; A61P 35/00

(57) ABSTRACT

The present invention relates to a superparamagnetic gold nanoparticle cluster-protein nanoparticle fusion body for magnetic resonance imaging and magnetic thermotherapy. According to the present invention, a superparamagnetic gold nanoparticle cluster-protein nanoparticle fusion body which has target directionality and a high density of ultrafine gold nanoparticles uniformly coupled to the surface of protein nanoparticles can be fabricated with neither a separate surface stabilization process nor a separate target directionality conferring process. Hence, the superparamagnetic gold nanoparticle cluster-protein nanoparticle fusion body according to the present invention is superior to conventional gold nanoparticles in terms of biocompatibility and has excellent target directionality as well as being identified to have a temperature elevation potential in an alternating magnetic field and a functionality as a T2-MRI contrast medium thanks to the superparamagnetism property of the ultrafine gold nanoparticles.

### CROSS-REFERENCE TO RELATED APPLICATION

This application is a continuation in part of U.S. patent application Ser. No. 16/628,773 filed on Jan. 6, 2020, is a U.S. national stage application of International Application No. PCT/KR2018/007556 filed on Jul. 4, 2018, which claims the benefit of Korean Patent Application No. 10-2017-0084793 filed on Jul. 4, 2017, in the Korean Intellectual Property Office, the entire disclosures of which are incorporated herein by reference for all purposes.

### BACKGROUND

### 1. Field of the Invention

The present invention relates to a superparamagnetic gold nanoparticle cluster-protein nanoparticle hybrid for magnetic resonance imaging and magnetic hyperthermia.

### 2. Discussion of Related Art

Despite advances in modem medicine, patients who undergo radiotherapy or drug-based therapy suffer from cytotoxicity caused by systemic administration of non-targeting drugs and associated side effects due to cytotoxicity, or side effects such as mutation or death of normal cells caused by radiotherapy. Further, when a diagnostic imaging technique is used in order to diagnose the corresponding disease, side effects such as normal organ damage due to accumulation of existing iron oxide-based contrast agents in the body are problematic. However, if a drug such as a therapeutic agent or a contrast agent is specifically delivered to a desired target tissue, it is possible to diagnose and treat a disease without any side effects on normal cells or normal tissues, and thus, studies for drug targeting have been actively conducted as a means for diagnosis and treatment without current side effects.

For drug targeting, methods of binding antibodies or peptides, specific ligands, or polymers that are specific to tissues or cells that are primarily intended for drug delivery are bound to a drug by chemical or physical method have been used. However, due to the inherent physicochemical properties of drugs, it is definitely not easy to bind antibodies, peptides, ligands, and the like, and problems affecting the pharmacological properties, biotoxicity, and the like of the drug may occur when a binder, a stabilizer, or the like for chemical or physical binding is used. Therefore, there is a continuing technical need for a method for drug targeting, which does not cause these problems.

Meanwhile, as one of the methods of killing target disease (particularly cancer) pathogenic cells by delivering a drug in a target-oriented manner, a method for delivering nanometer-scale ultrafine gold nanoparticles, followed by magnetic hyperthermia may be an effective treatment means. Since the ultrafine gold nanoparticles having the corresponding size have an inherent superparamagnetic property, the ultrafine gold nanoparticles can also function as a contrast agent for magnetic resonance imaging. Gold nanoparticles, at a size of several tens of nanometers, exhibit inherent optical or magnetic properties due to the quantization of surface electrons and based on these functionalities, gold nanoparticles are likely to be utilized variously in the fields such as single-electron devices, chemical sensors, biosensors, and drug delivery and catalysts.

However, when a conventional method such as chemical reduction reactions is used in order to synthesize ultrafine gold nanoparticles, metal compounds, solvents, reducing agents, or stabilizers are generally required. These chemical additives may cause toxicity, which presents a main limitation in utilizing gold nanoparticles in the medical field.

Further, in order to fabricate stable gold nanoparticles, surface modification of gold nanoparticles is definitely required. This is because when gold nanoparticles are not subjected to surface modification, aggregation occurs due to the structural instability upon a change in pH or concentrating gold nanoparticles to high concentration, and as a result, a case where the size and shape of particles are changed occurs. In addition, when gold nanoparticles are used for disease diagnosis and treatment, antibodies or targeting peptides need to be exposed at the surface of gold nanoparticles for the drug targeting previously described using chemical bonds, and in this case, since the corresponding material is irregularly bound to the surface of gold nanoparticles, there is a limitation in uniformly displaying this at high density.

In general, a material, which serves as a condensation nucleus of gold ions such as sodium citrate during the synthesis of gold nanoparticles is used, and it has been reported that when a peptide including an amino acid providing the standard reduction potential at which gold ions may be reduced at a pH of 7.0 or more is used, gold ions are reduced, and thus gold nanoparticles may be synthesized on the surface of the peptide (Korean Patent Application Laid-Open No. 2012-0052501). However, the technology reported in the Korean Patent Application Laid-Open No. 2012-0052501 only aggregates gold nanoparticles around a well-known gold affinity protein or gold ion adsorbable peptide by reacting a gold precursor with the gold affinity protein or gold ion adsorbable peptide, so that it is difficult to control the shape or size of gold nanoparticles, and additional chemical bonding is required to confer target aiming.

### SUMMARY OF THE INVENTION

The present invention has been devised to solve the conventional problems as described above, and the present inventors fabricated a superparamagnetic gold nanoparticle cluster-protein nanoparticle hybrid of the present invention by forming superparamagnetic ultrafine gold nanoparticles on recombinant HBV capsid protein nanoparticles. In addition, the present inventors confirmed the target-oriented magnetic hyperthermia of and the contrast effects of magnetic resonance imaging of the superparamagnetic gold nanoparticle cluster-protein nanoparticle hybrid, thereby completing the present invention based on this.

Thus, an object of the present invention is to provide a superparamagnetic gold nanoparticle cluster-protein nanoparticle hybrid having excellent temperature elevation potential and contrast effects for magnetic resonance imaging due to a magnetic field.

Another object of the present invention is to provide a method for treating cancer using the superparamagnetic gold nanoparticle cluster-protein nanoparticle hybrid for magnetic hyperthermia.

Still another object of the present invention is to provide a method for magnetic resonance imaging using the superparamagnetic gold nanoparticle cluster-protein nanoparticle hybrid for magnetic resonance imaging contrast.

To achieve the objects of the present invention as described above, the present invention provides a superparamagnetic gold nanoparticle cluster-protein nanoparticle hybrid including recombinant hepatitis B virus (HBV) capsid protein nanoparticles and a superparamagnetic gold nanoparticle cluster formed on the protein nanoparticles, in which the superparamagnetic gold nanoparticles have a diameter of 1 nm to 4 nm.

The present invention also provides a method for fabricating a superparamagnetic gold nanoparticle cluster-protein nanoparticle hybrid, the method including:

adding a gold precursor to recombinant hepatitis B virus (HBV) capsid protein nanoparticles including a gold ion adsorbable peptide and a superparamagnetic inducing peptide to adsorb the gold precursor on the recombinant hepatitis B virus (HBV) capsid protein nanoparticles; and

reducing gold ions of the gold precursor adsorbed on the recombinant hepatitis B virus (HBV) capsid protein nanoparticles to form superparamagnetic gold nanoparticles on the recombinant hepatitis B virus (HBV) capsid protein nanoparticles,

wherein the superparamagnetic gold nanoparticles have a diameter of 1 nm to 4 nm.

The present invention also provides a medical composition for magnetic hyperthermia, the composition including the superparamagnetic gold nanoparticle cluster-protein nanoparticle hybrid and a pharmaceutically acceptable carrier.

The present invention also provides a contrast agent composition for magnetic resonance imaging (MRI), the composition including the superparamagnetic gold nanoparticle cluster-protein nanoparticle hybrid and a pharmaceutically acceptable carrier.

According to the present invention, a superparamagnetic gold nanoparticle cluster-protein nanoparticle hybrid which has target aiming and a high density of ultrafine gold nanoparticles uniformly coupled to the surface of coupled to the surface of protein nanoparticles can be fabricated with neither a separate surface stabilization process nor a separate target aiming conferring process. The superparamagnetic gold nanoparticle cluster-protein nanoparticle hybrid according to the present invention is superior to conventional gold nanoparticles in terms of biocompatibility and has excellent target aiming as well as being identified as having a temperature elevation potential in an alternating magnetic field and a functionality as a T2-MRI contrast agent thanks to the superparamagnetic property of the ultrafine gold nanoparticles. Therefore, the superparamagnetic gold nanoparticle cluster-protein nanoparticle hybrid according to the present invention can be utilized as a core technology in the fields of magnetic hyperthermia and magnetic resonance imaging contrast agents.

### BRIEF DESCRIPTION OF THE DRAWINGS

FIG. 1 is a schematic view of a fabrication process and structure of a superparamagnetic gold nanoparticle cluster-protein nanoparticle hybrid (SPAuNC).

FIG. 2 is a schematic view of expression vectors for fabricating a recombinant hepatitis B virus (HBV) capsid protein.

FIG. 3 is a set of results including (A) TEM imaging results of, (B) dynamic light scattering (DLS) results of, and (C) energy-dispersive X-ray spectroscopy (EDX) results of an ultrafine (SPAuNC) or fine (DAuNC) gold nanoparticle cluster-protein nanoparticle hybrid.

FIG. 4 is a result confirming the correlation between the amount of protein nanoparticles of an ultrafine (SPAuNC) or fine (DAuNC) gold nanoparticle cluster-protein nanoparticle hybrid and the amount of surface gold nanoparticles (red solid line: the ultrafine gold nanoparticle cluster-protein nanoparticle hybrid, blue solid line: fine gold nanoparticle cluster-protein nanoparticle hybrid).

FIG. 5 is a result confirming the elevation of temperature over alternating magnetic field treatment time of an ultrafine (SPAuNC) or fine (DAuNC) gold nanoparticle cluster-protein nanoparticle hybrid, superparamagnetic iron oxide nanoparticles, gold nanoparticles (5, 20, and 40 nm), and HBV capsid nanoparticles.

FIG. 6 is an EPR spectrum result for the ultrafine gold nanoparticle cluster-protein nanoparticle hybrid (SPAuNC).

FIGS. 7A to 7C are magnetization curves of (A. B) an ultrafine (SPAuNC) or (C) fine (DAuNC) gold nanoparticle cluster-protein nanoparticle hybrid over temperature, measured through SQUID.

FIG. 8 is (A) the XPS measurement result for the 4f orbital of gold atoms in gold nanoparticles on the surface of chloro(trimethyl phosphine) gold (I) and an ultrafine gold nanoparticle cluster-protein nanoparticle hybrid (SPAuNC), (B) the XPS measurement result for the 2p orbital of phosphorus atoms in gold nanoparticles on the surface of chloro(trimethyl phosphine) gold (I) and an ultrafine gold nanoparticle cluster-protein nanoparticle hybrid (SPAuNC), and (C) the XPS measurement result for the is orbital of oxygen atoms in hexatyrosine on the surface of HBV capsid nanoparticles and an ultrafine gold nanoparticle cluster-protein nanoparticle hybrid (SPAuNC).

FIG. 9 is a schematic view briefly illustrating a process in which gold ions are reduced on the surface of the ultrafine gold nanoparticle cluster-protein nanoparticle hybrid (SPAuNC) and a process of acquiring the superparamagnetic property.

FIG. 10 illustrates results of comparing the change in target aiming of the ultrafine gold nanoparticle cluster-protein nanoparticle hybrid (SPAuNC) in the presence and absence of an EGFR-targeting peptide and the target aiming of superparamagnetic iron oxide nanoparticles, for an EGFR-overexpressing breast cancer cell line (MDA-MB-468 cell line).

FIG. 11 is (A) a result of verifying stability by injecting an ultrafine gold nanoparticle cluster-protein nanoparticle hybrid (SPAuNC) at each concentration into an EGFR-overexpressing breast cancer cell line (MDA-MB-468 cell line) without being treated with an alternating magnetic field, and then performing a cell viability analysis over the passage of time, and (B) a result of injecting an ultrafine (SPAuNC) or fine (DAuNC) gold nanoparticle cluster-protein nanoparticle hybrid and HBV capsid nanoparticles into an EGFR-overexpressing breast cancer cell line (MDA-MB-468 cell line), and then treating the resulting mixture with an alternating magnetic field, and analyzing cell viability over time.

FIG. 12 is (A) a result of verifying the ultrafine gold nanoparticle cluster-protein nanoparticle hybrid (SPAuNC) as a T1 or T2 contrast agent, and (B) a result of analyzing the T2 contrast effect over concentration of the ultrafine nanoparticle cluster-protein nanoparticle hybrid.

FIG. 13 is (A) a result of confirming the in vivo distribution of the administered ultrafine gold nanoparticle cluster-protein nanoparticle hybrid (SPAuNC) through near-infrared fluorescence imaging over the passage of time, and (B) a result of quantifying the fluorescence amount measured at a cancer occurring site in the presence and absence of an EGFR-targeting peptide over the passage of time, for an animal model in which EGFR-overexpressing breast cancer cells (MDA-MB-468 cell line) are implanted subcutaneously.

FIG. 14 is (A) a result of confirming the distribution of the administered ultrafine gold nanoparticle cluster-protein nanoparticle hybrid (SPAuNC) in each organ (the liver, the lungs, the spleen, the kidneys, and the heart) through near-infrared fluorescence imaging over the passage of time, and (B) a result of confirming the distribution of the ultrafine gold nanoparticle cluster-protein nanoparticle hybrid in liver normal tissues and cancer tissues through confocal immunofluorescence imaging, for an animal model in which EGFR-overexpressing breast cancer cells (MDA-MB-468 cell line) are implanted in the liver.

FIG. 15 is, 1) when the ultrafine gold nanoparticle cluster-protein nanoparticle hybrid (SPAuNC) is administered to an animal model in which EGFR-overexpressing breast cancer cells (MDA-MB-468 cell line) are implanted subcutaneously and the animal model is treated with an alternating magnetic field and 2) when the animal model is treated only with an alternating magnetic field without any material treatment, (A) a result of confirming the change in size of cancer (tumor volume) over the passage of time, and (B) a result of quantifying the change in cancer size and body weight over the passage of time (a: the case where the ultrafine gold nanoparticle cluster-protein nanoparticle hybrid (SPAuNC) is administered to the animal model and the animal model is treated with an alternating magnetic field, b: the case where the animal model is treated only with an alternating magnetic field without any material treatment).

FIG. 16 is, when the ultrafine gold nanoparticle cluster-protein nanoparticle hybrid (SPAuNC) is administered to an animal model in which EGFR-overexpressing breast cancer cells (MDA-MB-468 cell line) are implanted in the liver, and the animal model is treated with an alternating magnetic field, (A) a result of confirming the therapeutic effect by extracting the liver, and (B) a result of confirming whether normal liver tissues are damaged and the therapeutic effect by staining the overall liver tissue with H&E.

FIG. 17 is a result of confirming whether normal organs (the liver, the lungs, the heart, the spleen, the pancreas, and the kidneys) are damaged by administering the ultrafine nanoparticle cluster-protein nanoparticle hybrid (SPAuNC) to an animal model in which EGFR-overexpressing breast cancer cells (MDA-MB-468 cell line) are implanted subcutaneously, treating the animal model with an alternating magnetic field, and then staining the normal organs with H&E.

FIG. 18 is a set of images of capturing magnetic resonance images over the passage of time after the ultrafine gold nanoparticle cluster-protein nanoparticle hybrid (SPAuNC) is administered to an animal model in which EGFR-overexpressing breast cancer tissues (MDA-MB-468 cell line) are implanted subcutaneously and the animal model is treated with an alternating magnetic field.

FIG. 19 is (A) an image of capturing magnetic resonance images over the passage of time after the ultrafine gold nanoparticle cluster-protein nanoparticle hybrid is administered to an animal model in which EGFR-overexpressing breast cancer cells (MDA-MB-468 cell line) are implanted in the liver, and the animal model is treated with an alternating magnetic field, and (B) a set of images of capturing magnetic resonance images over the passage of time after the ultrafine gold nanoparticle cluster-protein nanoparticle hybrid is administered to the animal model and the animal model is treated with an alternating magnetic field.

FIG. 20 is a result of comparing whether gold nanoparticles in urine are excreted ex vivo by quantifying the gold nanoparticles in urine for the ultrafine gold nanoparticle cluster-protein nanoparticle hybrid (SPAuNC) and 20 nm gold nanoparticles.

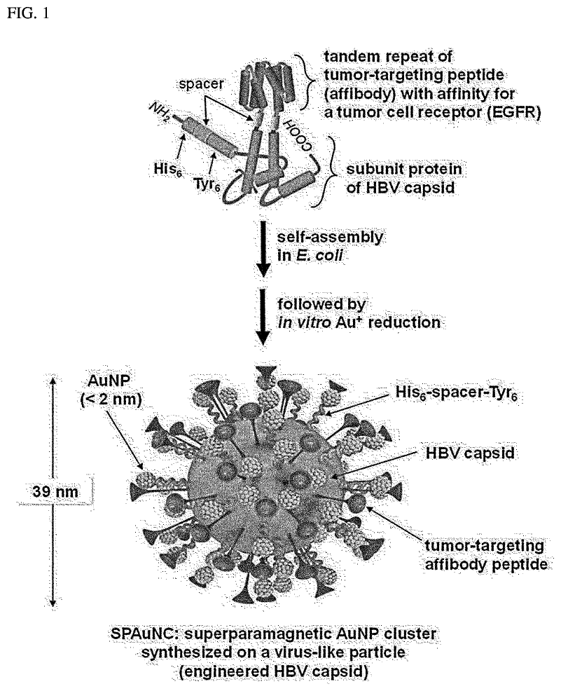

### DETAILED DESCRIPTION OF EXEMPLARY EMBODIMENTS

Hereinafter, the configuration of the present invention will be described in detail.

The present invention relates to a superparamagnetic gold nanoparticle cluster-protein nanoparticle hybrid including recombinant hepatitis B virus (HBV) capsid protein nanoparticles and a superparamagnetic gold nanoparticle cluster formed on the protein nanoparticles, in which the superparamagnetic gold nanoparticles have a diameter of 1 nm to 4 nm.

The present invention has a technical significance, in that the superparamagnetic gold nanoparticle cluster-protein nanoparticle hybrid of the present invention has a superparamagnetic property derived from gold nanoparticles having an ultrafine (diameter: 1 nm to 4 nm) size. As used herein, the term “superparamagnetic” refers to a property that when there is no external magnetic field, the magnetic moments of nanoparticles are randomized by heat energy, and thus magnetic properties are not observed, whereas when an external magnetic field is applied, the magnetic moments of nanoparticles are aligned in a predetermined direction, and thus strong magnetic properties are exhibited. In particular, when there is no external magnetic field, superparamagnetic nanoparticles may maintain a stable colloidal state because the nanoparticles rarely interact, so that the superparamagnetic nanoparticles may be extended to and applied in the field of biomedicine, particularly, magnetic hyperthermia, magnetic resonance imaging technology, and the like.

As superparamagnetic nanoparticles currently applied to the field of biomedicine, iron oxide nanoparticles have been developed, but side effects such as damage to normal organs due to the accumulation of iron oxide-based materials in the body have been reported. In contrast, the present invention has another technical significance, in that gold nanoparticles having an ultrafine size (diameter: 1 nm to 4 nm) included in the superparamagnetic cluster-protein nanoparticle hybrid of the present invention may be excreted ex vivo within a short period of time after being administered, and thus have excellent biocompatibility or stability.

In the present invention, the hepatitis B virus (HBV) capsid protein nanoparticle may refer to a spherical protein nanoparticle formed by self-assembling 180 or 240 capsid monomers, or each capsid monomer forming the protein nanoparticles. Since each capsid protein is expressed in the form of a fusion protein including a gold ion adsorbable peptide, and the like, and then self-assembled to form nanoparticles, it is possible to reproducibly mass-produce particles exhibiting a very uniform particle size distribution. In addition, since the capsid protein is a biocompatible material that may be decomposed after being used in vivo, there is no toxicity problem due to remaining nanoparticles. Further, since the recombinant hepatitis B virus (HBV) capsid protein of the present invention imparts a gold ion adsorbable peptide, a superparamagnetic inducing peptide, a target-oriented peptide, and the like to protein nanoparticles by a genetic engineering method instead of a chemical method, there are advantages in that the surface display frequency and position thereof may be controlled as desired and the modification of biological activity may be minimized.

In one embodiment of the present invention, the recombinant HBV capsid protein includes a gold ion adsorbable peptide.

The gold ion adsorbable peptide has characteristics of adsorbing a gold ion provided in the form of a gold precursor. The gold ion adsorbable peptide may be used without limitation in sequence as long as the gold ion adsorbable peptide has gold ion adsorption characteristics. In an embodiment, the gold ion adsorbable peptide may include an amino acid sequence including a plurality of histidines (Hₙ, n≥2), but is not limited thereto. In one embodiment, the gold ion adsorbable peptide may be introduced at the N-terminus of the recombinant HBV capsid protein. The introduction of the gold ion adsorbable peptide at the N-terminus of the recombinant HBV capsid protein may be advantageous in terms of display at the surface of the protein nanoparticles or control of the size of gold nanoparticles. In an example of the present invention, the gold ion adsorbable peptide was displayed at high density on the surface of the recombinant HBV capsid protein nanoparticles by expressing hexahistidine as the gold ion adsorbable peptide at the N-terminus of the HBV capsid protein.

In one embodiment of the present invention, the recombinant hepatitis B virus (HBV) capsid protein may further include a superparamagnetic inducing peptide. The superparamagnetic inducing peptide may be used regardless of type as long as the peptide has a sequence allowing charge transfer so as to induce a superparamagnetic property in gold nanoparticles. As one embodiment, the superparamagnetic inducing peptide may include an amino acid sequence including any one or more selected from the group consisting of a plurality of tyrosines (Yₙ, n≥2), threonines (Tₙ, n≥2), serines (Sₙ, n≥2), and cysteines (Cₙ, n≥2). Additionally, the upper limit of the above-described n may be, for example, 50 to 3, but is not limited thereto. In an example of the present invention, a superparamagnetic property was induced in gold nanoparticles through charge transfer using hexatyrosine as the superparamagnetic inducing peptide.

In the present invention, the superparamagnetic gold nanoparticles formed on the recombinant hepatitis B virus (HBV) capsid protein nanoparticles may be formed from a gold precursor. The superparamagnetic gold nanoparticles are synthesized by reducing gold ions of a gold precursor adsorbed onto the gold ion adsorbable peptide.

As the gold precursor, well-known gold precursors such as chloro(trimethylphosphine) gold (AuClP(CH₃)₃), potassium tetrachloroaurate (II)(KAuCl₄), sodium tetrachloroaurate (NaAuCl₄), chloroauric acid (HAuCl₄), gold sodium bromide (NaAuBr₄), gold chloride (AuCl), gold chloride (III)(AuCl₃), or gold bromide (AuBr₃) may be applied.

For a reaction of the gold precursor with the recombinant self-assembling protein nanoparticles, the reaction is initiated by adding a reducing agent. This is because the protein itself has high structural stability, and thus it is difficult to form gold particles even though gold ions are added unless the strong reducing power of the reducing agent is provided. Although the reducing agent is used, the present invention may allow the reaction to be performed under room temperature conditions instead of high temperature conditions, and the produced superparamagnetic gold nanoparticle cluster-protein nanoparticle hybrid has very high structural stability without any additional surface treatment.

Further, the reaction has an advantage in that it is possible to regulate the size of gold nanoparticles formed on the surface of protein nanoparticles by the type and amount of reducing agent, and the reaction time. Ultrafine gold nanoparticles having a superparamagnetic property may be formed using this property. Specifically, ultrafine gold nanoparticles formed on the recombinant hepatitis B virus (HBV) capsid protein nanoparticles may have a diameter of, for example, 1 nm to 4 nm. In one embodiment of the present invention, ultrafine gold nanoparticles having a diameter of 1.4 nm were formed by adding 0.01 to 0.1 mol of a reducing agent NaBH₄ to 1 mg of HBV capsid protein nanoparticles and reacting them for 15 minutes. When electron movement (charge transfer) occurs from gold atoms on the surface of ultrafine gold nanoparticles to oxygen atoms of tyrosine and phosphine phosphorous, the superparamagnetic property of ultrafine gold nanoparticles is expressed.

In one embodiment of the present invention, the recombinant hepatitis B virus (HBV) capsid protein may further include a target-oriented peptide.

The target-oriented peptide refers to a peptide capable of binding to the surface of cells such as cancer cells or inflammatory cells, such that the superparamagnetic gold nanoparticle cluster-protein nanoparticle hybrid according to the present invention can move to a target site in vivo, which requires treatment or diagnosis, such as cancer cells or inflammatory cells.

For the purpose of the present invention, the target-oriented peptide may be introduced so as to be displayed on the surface of recombinant self-assembling protein nanoparticles. In one embodiment of the present invention, the target-oriented peptide may be introduced at the spike site of the recombinant HBV capsid protein. More specifically, the target-oriented peptide may be introduced into a part between 1-78 amino acid positions and 81-149 amino acid positions, of the recombinant HBV capsid protein. An example of the present invention shows that the specific targeting of superparamagnetic gold nanoparticle cluster-protein nanoparticle hybrid is possible by introducing the target-oriented peptide at the spike site of the recombinant HBV capsid protein.

In addition, any target-oriented peptide can be used as long as the superparamagnetic gold nanoparticle cluster-protein nanoparticle hybrid can target a desired tissue or cell so as to be used suitably for the purpose. In one embodiment of the present invention, the target-oriented peptide may be a target-oriented peptide against cancer cells. For example, the target-oriented peptide against cancer cells may target any one selected from the group consisting of integrin, vimentin, human fibronectin extradomain B (EDB), interleukin-4 (IL-4), human epidermal growth factor receptor 2(HER-2). CD20, poliovirus receptor (PVR), VISTA, 4-1BBL, Galectin-9, Adenosine A2a receptor, CD80, CD86, ICOS, ICOSL, BTLA, OX-40L, CD155, BCL2, MYC, PP2A, BRD1, BRD2, BRD3, BRD4, BRDT, CBP, E2F1, MDM2, MDMX, PPP2CA, PPM1D, STAT3, IDH1, PD1, CTLA4, PD-L1, PD-L2, LAG3, TIM3, TIGIT, BTLA, SLAMF7, 4-1BB, OX-40, ICOS, GITR, ICAM-1, BAFFR, HVEM, LFA-1, LIGHT, NKG2C, SLAMF7, NKp80, LAIR1, 2B4, CD2, CD3, CD16, CD20, CD27, CD28, CD40L, CD48, CD52, EGFR family, AXL, CSF1R, DDR1, DDR2, EPH receptor family, FGFR family. VEGFR family, IGF1R, LTK, PDGFR family, RET, KIT, KRAS, NTRK1 and NTRK2, but is not limited thereto. In an example of the present invention, a target-oriented peptide targeting EGFR having specificity in human breast cancer cells was used, but the target-oriented peptide of the present invention is not limited thereto.

In one embodiment of the present invention, the recombinant HBV capsid protein may further include a linker peptide between a gold ion adsorbable peptide and a superparamagnetic inducing peptide. The linker peptide is a non-reactive peptide including a plurality of glycines, may simultaneously block the possibility that gold ions may be excessively reduced while the gold particles may be sufficiently grown by securing the space between a hexahistidine peptide and a superparamagnetic inducing peptide in an appropriate size, and provides structural stability.

In another embodiment of the present invention, the recombinant HBV capsid protein may further include another linker peptide between the linker peptide and the superparamagnetic inducing peptide. The linker peptide may have a length capable of securing an appropriate space between the superparamagnetic inducing peptide and the existing linker peptide. Accordingly, the length of the linker peptide may vary depending on the types and sizes of the superparamagnetic inducing peptide and the existing linker peptide. For example, the linker peptide may be a peptide including 5 to 20, for example, 5 to 15 amino acids.

In still another embodiment of the present invention, in order to facilitate the isolation and purification, the recombinant HBV capsid protein may further include a peptide for isolation and purification, such as a FLAG (DYKDDDDK: SEQ ID No. 16) tag and a GST tag.

The present invention also relates to a method for fabricating a superparamagnetic gold nanoparticle cluster-protein, the method including:

adding a gold precursor to recombinant hepatitis B virus (HBV) capsid protein nanoparticles including a gold ion adsorbable peptide and a superparamagnetic inducing peptide to adsorb the gold precursor on the recombinant hepatitis B virus (HBV) capsid protein nanoparticles; and

reducing gold ions of the gold precursor adsorbed on the recombinant hepatitis B virus (HBV) capsid protein nanoparticles to form superparamagnetic gold nanoparticles on the recombinant hepatitis B virus (HBV) capsid protein nanoparticles,

wherein the superparamagnetic gold nanoparticles have a diameter of 1 nm to 4 nm.

The reduction of the gold ions may be a reaction performed by adding 0.005 to 0.015 mol of a reducing agent to 1 mg of the recombinant hepatitis B virus (HBV) capsid protein, but is not limited thereto, and the reaction time may be 2 to 30 minutes, for example, 5 to 20 minutes. In an example of the present invention, NaBH₄ was used as a reducing agent, but any reducing agent may be used without limitation as long as the reducing agent is well-known as a reducing agent of gold ions.

Meanwhile, in one embodiment, the present invention exhibited remarkable temperature elevation and excellent T2 contrast effects for magnetic resonance imaging in an alternating magnetic field along with the specific target aiming of the superparamagnetic gold nanoparticle cluster-protein nanoparticle hybrid.

Thus, as another aspect of the present invention, the present invention provides a medical use of the superparamagnetic gold nanoparticle cluster-protein nanoparticle hybrid for magnetic hyperthermia; a medical composition for magnetic hyperthermia, including the superparamagnetic gold nanoparticle cluster-protein nanoparticle hybrid and a pharmaceutically acceptable carrier; and a magnetic hyperthermia method including: administering the superparamagnetic gold nanoparticle cluster-protein nanoparticle hybrid to a subject.

Since the medical use and the like of the superparamagnetic gold nanoparticle cluster-protein nanoparticle hybrid for magnetic hyperthermia use the technical configuration as described for the above-described superparamagnetic gold nanoparticle cluster-protein nanoparticle hybrid, the description of content common to both of them will be omitted in order to avoid the excessive complexity of the present specification.

As used herein, the term “magnetic hyperthermia” refers to a technology that treats a lesion site using heat generated by applying an external magnetic field, and is usually applied to target and treat cancer cells which are killed particularly at a temperature of 42° C. or more. Necessary conditions of the nanoparticles which may be applied to the magnetic hyperthermia are as follows: 1) generate therapeutically effective heat by an external magnetic field, 2) decompose or release the nanoparticles without accumulating the nanoparticles in vivo, and 3) minimize damage to normal tissues through delivery to a specific lesion site. In this respect, the composition in the present invention 1) may generate heat sufficient to kill cancer cells due to excellent superparamagnetic properties, 2) may allow ultrafine-sized gold nanoparticles to be released rapidly through urine, and 3) may effectively display a target-oriented peptide due to the structural characteristics of the recombinant HBV capsid protein, thereby contributing to the maximization of effects of magnetic hyperthermia.

Further, as described below, the composition of the present invention may be utilized as a contrast agent for magnetic resonance imaging, and thus, the present invention has still another technical significance, in that the composition of the present invention can achieve real-time monitoring of the magnetic hyperthermia effects without being limited to simply providing therapeutic effects.

In relation to the conditions of the alternating magnetic field applied to generate heat of the superparamagnetic gold nanoparticle cluster-protein nanoparticle hybrid according to the present invention, the frequency may be, for example, 300 kHz to 450 kHz, or 330 kHz to 390 kHz, the electrical energy may be, for example, 5 kW to 15 kW, or 8 kW to 12 kW, and the treatment time may be, for example, 5 minutes to 20 minutes, or 5 minutes to 15 minutes, but may be appropriately changed by reflecting characteristics, severity, or the like of the disease.

Furthermore, the magnetic hyperthermia according to the present invention may be used in cell-level treatment without limitation, and the cells to be treated are changed depending on the above-described target-oriented peptide. In one embodiment, the present invention may be used for targeted treatment, that is, cancer treatment, but is not limited thereto.

Further, as still another aspect of the present invention, the present invention provides a medical use of the superparamagnetic gold nanoparticle cluster-protein nanoparticle hybrid for magnetic resonance imaging contrast; a contrast agent composition for magnetic resonance imaging (MRI), including the superparamagnetic gold nanoparticle cluster-protein nanoparticle hybrid and a pharmaceutically acceptable carrier; and a magnetic resonance imaging method including: administering the superparamagnetic gold nanoparticle cluster-protein nanoparticle hybrid to a subject.

Since the medical use and the like of the superparamagnetic gold nanoparticle cluster-protein nanoparticle hybrid for magnetic resonance imaging contrast use the technical configuration as described for the above-described superparamagnetic gold nanoparticle cluster-protein nanoparticle hybrid, the description of content common to both of them will be omitted in order to avoid the excessive complexity of the present specification.

As used herein, the term “magnetic resonance imaging (MRI)” refers to a method for obtaining anatomical, physiological, and biochemical images of the body using a phenomenon in which the spin of hydrogen atoms relaxes in a magnetic field, and is currently a technology capable of imaging a body organ of a living human or animal non-invasively and in real time.

As used herein, the term “contrast agent” refers to an external material for increasing the contrast degree of the magnetic resonance imaging, and these contrast agents generally have superparamagnetic or paramagnetic characteristics.

Specifically, the contrast agents are classified into a positive contrast agent (T1 contrast agent) exhibiting relatively high contrast as compared to imaging signals of the body site and a negative contrast agent (T2 contrast agent) exhibiting relatively low contrast as compared to imaging signals of the body site, paramagnetic nanoparticles are used in the former case, and superparamagnetic nanoparticles are used in the latter case. Accordingly, the composition according to the present invention may be utilized as a magnetic resonance imaging T2 contrast agent.

The magnetic resonance imaging method according to the present invention may be performed by adopting technologies known in the art, in addition to using the superparamagnetic gold nanoparticle cluster-protein nanoparticle hybrid. In an example of the present invention, T2 MRI imaging is performed using a magnetic resonance imaging apparatus having an alternating magnetic field intensity of 4.7-T, but is not limited thereto.

In addition, the magnetic resonance imaging according to the present invention may be used without limitation for imaging at cell level, and the cells of interest are changed depending on the above-described target-oriented peptide. In one embodiment, the present invention may be used for imaging of cancer cells, that is, for monitoring a cancer tissue, but is not limited thereto.

Meanwhile, a pharmaceutically acceptable carrier used for the medical composition for magnetic hyperthermia or the contrast agent composition according to the present invention includes a carrier and a vehicle typically used in the medical field.

The pharmaceutically acceptable carrier includes an ion exchange resin, alumina, aluminum stearate, lecithin, a serum protein, a buffer material, water, a salt or electrolyte, colloidal silica, magnesium trisilicate, polyvinylpyrrolidone, a cellulose-based substrate, polyethylene glycol, sodium carboxymethyl cellulose, polyarylates, wax, polyethylene glycol, wool grease, but is not limited thereto. The composition of the present invention may additionally include a lubricant, a wetting agent, an emulsifier, a suspending agent, a preservative, and the like, in addition to the aforementioned ingredients.

As one embodiment of the present invention, the composition according to the present invention may be prepared as an aqueous solution for parenteral administration. For example, it is possible to use a buffer solution such as Hank's solution, Ringer's solution, or physically buffered saline. An aqueous injection suspension may have an added substrate capable of increasing the viscosity of the suspension, such as sodium carboxymethyl cellulose, sorbitol or dextran.

A preferred composition of the present invention may be in the form of a sterile injectable preparation as a sterile injectable aqueous or oily suspension. The suspension may be formulated according to the technology publicly known in the art using a suitable dispersant or wetting agent and a suspending agent. The sterile injectable preparation may also be a sterile injectable solution or suspension in a non-toxic parenterally acceptable diluent or solvent. Examples of a vehicle and a solvent that may be used include mannitol, water, Ringer's solution, and an isotonic sodium chloride solution. Furthermore, sterile, non-volatile oils are typically used as a solvent or suspending medium. A mild or non-volatile oil, including synthetic mono- or diglycerides, may be used for this purpose.

The present invention also relates to a method for treating cancer.

The method for treating cancer includes: administering the superparamagnetic gold nanoparticle cluster-protein nanoparticle hybrid comprising recombinant hepatitis B virus (HBV) capsid protein nanoparticles and a superparamagnetic gold nanoparticle cluster formed on the protein nanoparticles to a subject in need of treating cancer.

The term “subject” encompasses mammals and non-mammals. Examples of mammals include, but are not limited to, any member of the mammalian class: humans, nonhuman primates such as chimpanzees, and other apes and monkey species, farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In some embodiments of the methods and compositions provided herein, the mammal is a human or a nonhuman.

The administration may be performed via various routes. Non-limiting examples of the administration include oral (enteral) administration, non-oral administration (by injection), rectal administration, dermal administration, intradermal administration, intracranial administration, subcutaneous (SC) administration, intravenous (IV) administration, intramuscular (IM) administration, and intranasal administration.

The recombinant HBV capsid protein is the same as described above.

The recombinant hepatitis B virus (HBV) capsid protein may further include a target-oriented peptide for cancer cells. As the target-oriented peptide for cancer cells, those exemplified above may be used. In addition, any peptide having target-oriented peptide for cancer cells may be used.

The superparamagnetic gold nanoparticle cluster-protein nanoparticle hybrid may generate heat to kill cancer cells treated when an external magnetic field is applied. Accordingly, the gold nanoparticles may have, for example, a particle diameter of 1 nm to 4 nm, more specifically, 1.2 nm to 1.6 nm, in terms of ease of discharge, but is not limited thereto.

The superparamagnetic gold nanoparticle cluster-protein nanoparticle hybrid may generate heat to kill cancer cells treated when an external magnetic field is applied.

The magnetic field may be an alternating magnetic field. This may be applied under conditions in which the superparamagnetic gold nanoparticle cluster-protein nanoparticle hybrid can generate sufficient heat (eg, 40° C. or higher, 42° C. or higher, etc.) to kill cancer cells. Specifically, the frequency may be, for example, 300 kHz to 450 kHz, or 330 to 390 kHz, the amount of power may be, for example, 5 kW to 15 kW, or 8 to 12 kW, and the processing time may be, for example, 5 minutes to 20 minutes, or 5 minutes to 15 minutes, but is not limited thereto.

The benefits and features of the present application and the methods of achieving the benefits and features will become apparent with reference to exemplary embodiments to be described below in detail. However, the present invention is not limited to the exemplary embodiments to be disclosed below, but may be implemented in various other forms, and the present exemplary embodiments are only provided for rendering the disclosure of the present invention complete and for fully representing the scope of the invention to a person with ordinary skill in the technical field to which the present invention pertains, and the present invention will be defined only by the scope of the claims.

### <Example 1> Construction of Expression Vector for Biosynthesis of HBV Capsid-Derived Chimeric Nanoparticles

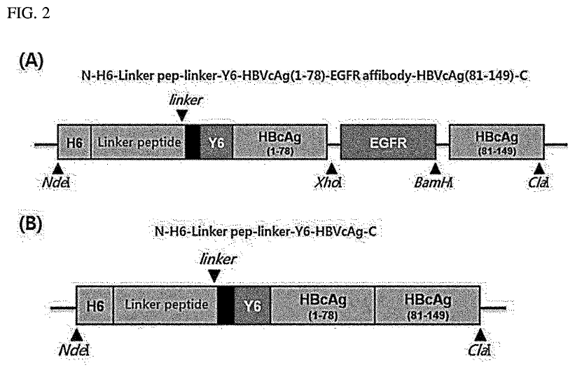

After extension PCR using a gene sequence of a HBV capsid protein (NCBI nucleotide accession number: 19010-2452 sequence among AF286594 sequence, SEQ ID No. 1) as a template using the primers 1 to 5 and a primer 6 shown in the following Table 1, two gene clones encoding the synthesis of N-NdeI-H6(hexahistidine)-LP (linker peptides)-Y6(hexatyrosine)-HBVcAg(1-78)-XhoI-C(SEQ ID No. 2) and N-BamHI-HBVcAg(81-149)-ClaI-C(SEQ ID No. 3) derived from a HBV core protein (HBVcAg) gene were obtained. Further, in order to replace P79A80 of HBVcAg with an epidermal growth factor receptor (EGFR) 1, 5′-XhoI-EGFR affibody-BamHI-3′ (SEQ ID No. 4) was prepared using PCR. A plasmid expression vector pT7-LP-Y6-EGFR affibody-HBcAg encoding the synthesis of N-H6-LP-Y6-HBVcAg(1-78)-EGFR affibody-HBVcAg(81-149)-C(SEQ ID No. 5) was constructed at a plasmid pT7-7 through a series of ligations of the gene clone (FIG. 2). All the prepared plasmid expression vectors were purified on an agarose gel, and then the sequence was confirmed through complete DNA sequencing.

The information on the primer sequence and template associated with preparation of HBV capsid-derived chimeric nanoparticles and a more detailed description in this regard are as follows (Table 1).

For a first part, extension PCR was performed on a primer sequence part 1 to a primer sequence part 5 in which a restriction enzyme NdeI was included along with primer sequence 6 including XhoI by employing a gene sequence of a HBV capsid protein (NCBI Nucleotide accession number: 1901-2452 sequence among AF286594 sequence, SEQ ID No. 1) as a template. As a result, a PCR product including a 5′-NdeI-H6-LP-Y6-HBV capsid protein (amino acid sequence 1-78)-XhoI-3′ (SEQ ID No. 2) was obtained.

2) For a second part, PCR was performed using a primer sequence part 7 in which BamHI was included and a primer sequence 8 including ClaI by employing a gene sequence of a HBV capsid protein as a template. As a result, a PCR product including a 5′-BamHI-HBV capsid protein (amino acid sequence 81-149)-ClaI-3′ (SEQ ID No. 3) was obtained.

3) For a third part, PCR was performed using primer sequence 9 including XhoI and primer sequence 10 including BamHI by employing an EGFR base sequence as a template. In order to prepare 5′-XhoI-EGFR affibody-BamHI-3′ (SEQ ID No. 4), primer sequences 9 and 10 were used.

An expression vector capable of synthesizing a functional group (hexatyrosine) derived from HBV capsid which may induce the reduction of gold ions and an EGFR affibody having a specific binding ability to human breast cancer cells was constructed by sequentially inserting the PCR products thus prepared into a pT7-7 vector (FIG. 2(A)).

4) In order to compare the binding ability of the EGFR affibody to cancer cells, extension PCR was performed on primer sequence Part 1 to primer sequence part 5 along with primer sequence 8 including ClaI, and an expression vector having a functional group (hexatyrosine) derived from HBV capsid capable of inducing the adsorption of gold ions was constructed by inserting the resulting PCR product into a pT7-7 vector, and was used as a control for the EGFR affibody (FIG. 2(B)).

TABLE 1

| | | |
|---|---|---|
| Primer 1 | 5′ NdeI- | CATATG CATCACCATCACCATCAC |
| (SEQ ID | H6-LP-Y6- | ATGGCGTCTAGTCTGCGT |
| No. 6) | HBcAg [symbol] (1) | |
| Primer 2 | 5′ NdeI- | ATGG [symbol] GTCTAGTCTGCGTCAGATT |
| (SEQ ID | H6-LP-Y6- | CTGGATTCTCAGAAAATGGAATGGCG |
| No. 7) | HBcAg [symbol] (2) | |
| Primer 3 | 5′ NdeI- | CAGAAAATGGAATGGCGTTCTAATGCG |
| (SEQ ID | H6-LP-Y6- | GGTGGCTCTGGTGGCGGAAGTGGG |
| No. 8) | HBcAg [symbol] (3) | |
| Primer 4 | 5′ NdeI- | GGTGGCGGAAGTGGGGGAGGCACTGGA |
| (SEQ ID | H6-LP-Y6- | GGTGGCGGCGGTGGG TACTATTAC |
| No. 9) | HBcAg [symbol] (4) | |
| Primer 5 | 5′ NdeI- | GGCGGTGGG TACTATTACTATTACTAT |
| (SEQ ID | H6-LP-Y6- | GACATTGACCCGTATAAAGA |
| No. 10) | HBcAg [symbol] (5) | |
| Primer 6 | 3′ XhoI- | CTCGAG GTCTTCCAAATTACTTCCCA |
| (SEQ ID | HBcAg78 | |
| No. 11) | | |
| Primer 7 | 5′ [symbol] - | GGATCC TCCAGGGAATTAGTAGTCAGC |
| (SEQ ID | HBcA [symbol] 81 | |
| No. 12) | | |
| Primer 8 | 3′ [symbol] - | ATCGAT TTAAACAACAGTAGTTTCCGG |
| (SEQ ID | HBcAg [symbol] 49 | AAGTGT |
| No. 13) | | |
| Primer 9 | 5′ XhoI- | CTCGAG GTGGATAACAAATTTAACAAA |
| (SEQ ID | EGFR | |
| No. 14) | | |
| Primer 10 | 3′ [symbol] - | GGATCC TTTCGGCGCCTGCGCATCGTT |
| (SEQ ID | EGFR | CAGTTTTTTCGCTTC |
| No. 15) | | |

[symbol]

indicates data missing or illegible when filed

### <Example 2> Biosynthesis of HBV Capsid-Derived Chimeric Nanoparticles

An E. coli strain BL21(DE3)[F-ompThsdSB(rB-mB-)] was transformed with the prepared expression vector, and ampicillin-resistant transformants were selected. The transformed E. coli was cultured in flasks (250 mL Erlenmeyer flasks, 37° C., 150 rpm) containing 50 mL of a Luria-Bertani (LB) medium (containing 100 mgL⁻¹ ampicillin). When medium turbidity (O.D 600) reached about 0.4 to 0.5, the expression of the recombinant gene was induced by adding isopropyl-O-D-thiogalactopyranosid (IPTG) (1.0 mM) thereto. After culturing at 20° C. for 16 to 18 hours, a bacterial cell precipitate was collected by centrifuging the cultured E. coli at 4,500 rpm for 10 minutes, and then suspended in 5 ml of a lysis solution (10 mM Tris-HCl buffer, pH 7.5, 10 mM EDTA) and disrupted using an ultrasonic cell disruptor (Branson Ultrasonics Corp., Danbury, Conn., USA). After disruption, centrifugation was performed at 13,000 rpm for 10 minutes, and then the supernatant and the insoluble aggregate were separated. Purification was performed according to Example 3 using the separated supernatant.

### <Example 3> Purification of HBV Capsid-Derived Chimeric Nanoparticles

In order to purify self-assembled EGFR protein fusion protein nanoparticles among the recombinant proteins expressed as described above, a three-step purification process was performed. First, 1) Ni2+-NTA affinity chromatography using binding of histidine fused to and expressed at the recombinant protein and nickel was performed, and then 2) in order to improve the efficiency of self-assembly of the recombinant protein, an aqueous solution including the recombinant protein was changed into a buffer for self-assembly (500 mM NaCl, 50 mM Tris-HCl, pH 7.0), and simultaneously, the recombinant protein was concentrated, using an ultracentrifugal filter (Amicon Ultra 100K, Millipore, Billerica, Mass.), and 3) finally, sucrose gradient ultracentrifugation was performed in order to separate only the self-assembled protein nanoparticles. Detailed description for each step is as follows.

### 1) Ni2+-NTA Affinity Chromatography

In order to purify the recombinant protein, the cell pellet was re-suspended in 5 mL of a lysis buffer (pH 8.0, 50 mM sodium phosphate, 300 mM NaCl, 20 mM imidazole) by collecting cultured E. coli by the same method described above, and cells were disrupted using an ultrasonic cell disruptor. After only the supernatant was separated by centrifuging the disrupted cell solution at 13,000 rpm for 10 minutes, each recombinant protein was separated using a Ni2+-NTA column (Qiagen, Hilden, Germany)(wash buffer: pH 8.0, 50 mM sodium phosphate, 300 mM NaCl, 80 mM imidazole/elution buffer: pH 8.0, 50 mM sodium phosphate, 300 mM NaCl, 200 mM imidazole).

### 2) Buffer Change for Promoting Self-Assembly, and Concentration

After 3 mL of the recombinant protein eluted by Ni2+-NTA affinity chromatography was loaded onto an ultracentrifugal filter (Amicon Ultra 100K, Millipore, Billerica, Mass.) and centrifuged at 5,000 g for 10 minutes, the upper part of the column was fully filled with a buffer for self-assembly (500 mM NaCl, 50 mM Tris-HCl pH 7.0), and then centrifugation at 5,000 g was conducted until 500 μl of the solution remained in the upper part of the column. After this procedure was three times repeated, a final volume was adjusted to 1 mL, and then the following step was conducted.

### 3) Sucrose Gradient Ultracentrifugation

After solutions having sucrose concentrations of 60%, 50%, 40%, 30%, 20%, and 10% were each prepared by adding sucrose at each concentration to the buffer for self-assembly, 2 mL of the sucrose solutions at each concentration (60 to 20%) was each loaded in a descending order of the sucrose concentration into an ultracentrifugation tube (Ultra-Clear 13.2 ml tube, Beckman). Finally, after 0.5 mL of the 10% sucrose solution was loaded, the 10% sucrose solution was filled with 1 ml of the recombinant protein present in the prepared buffer for self-assembly, and then ultracentrifugation was performed at 4° C. and 24,000 rpm for 16 hours (Ultracentrifuge L-90k, Beckman). After centrifugation, the upper layer (10 to 40% sucrose solution part) was removed carefully using a pipette and for the 50 to 60% sucrose solution part, the buffer of the recombinant protein was replaced using an ultracentrifugal filter and a buffer for self-assembly as specified in 2).

### <Example 4> Reduction of Gold Ions on Surface of Prepared HBV Capsid-Derived Chimeric Nanoparticles

Hexatyrosine, which can reduce gold ions, was bound to the N-terminus of the HBV capsid-derived chimeric nanoparticles obtained in Example 3.

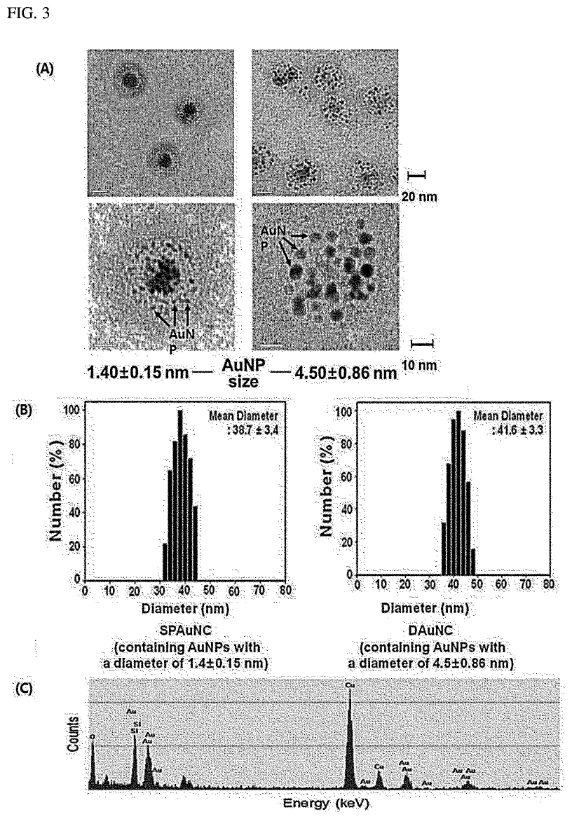

Chloro(trimethylphosphine) gold (I) (AuClP(CH₃)₃) was added to the HBV capsid-derived chimeric nanoparticles contained in a recombinant protein buffer with a pH of 7.0, the resulting mixture was reacted for 16 hours, followed by centrifugation at 4° C. and 13,000 rpm for 10 minutes. As a result of separating the supernatant, adding 0.01 mol of a reducing agent NaBH₄ to 1 mg of protein nanoparticles into the supernatant, and reacting the resulting mixture for 15 minutes, an ultrafine gold nanoparticle cluster-protein nanoparticle hybrid (SPAuNC)(left of FIG. 3(A)) in which ultrafine gold nanoparticles having a diameter of about 1.4 nm were uniformly synthesized on the surface was formed, and when 0.02 mol of the reducing agent NaBH₄ was added to 1 mg of protein nanoparticles and the resulting mixture was reacted for 50 minutes, it was possible to obtain a fine gold nanoparticle cluster-protein nanoparticle hybrid (DAuNC)(right of FIG. 3(A)) in which fine gold nanoparticles having a diameter of about 4.5 nm were uniformly synthesized on the surface (meanwhile, in the following present specification, SPAuNC and DauNC refer to an ultrafine or superparamagnetic gold nanoparticle cluster-protein nanoparticle hybrid and a fine or paramagnetic gold nanoparticle cluster-protein nanoparticle hybrid, respectively).

### <Example 5> Analysis of Structure of HBV Capsid-Derived Chimeric Nanoparticles

For the analysis of the structure of the purified ultrafine and fine gold-protein nanoparticle hybrids as described above, the hybrids were photographed using a transmission electron microscope (TEM). First, a purified protein sample which had not been stained was placed on carbon-coated copper electron microscope grids, and then naturally dried. In order to obtain stained images of the hybrids, electron microscope grids including the naturally dried sample were incubated with a 2% (w/v) aqueous uranyl acetate solution at room temperature for 10 minutes, and washed three to four times with distilled water. As a result of observing the images of ultrafine and fine gold-protein nanoparticle hybrids using a Philips Technai 120 kV electron microscope, first, it could be seen through TEM images that ultrafine gold nanoparticles having a diameter of about 1.4 nm uniformly formed a cluster in the form of raspberry on the surface (left of FIG. 3(A)), and through DLS analysis, an ultrafine gold nanoparticle cluster-protein nanoparticle hybrid (SPAuNc) (38.7±3.4 nm) including ultrafine gold nanoparticles (1.4±0.2 nm) could be identified (left of FIG. 3(B)). In addition, it could be seen through TEM images that fine gold nanoparticles having a diameter of about 4.5 nm uniformly formed a cluster on the surface (left of FIG. 3(A)), and through DLS analysis, a fine gold nanoparticle cluster-protein nanoparticle hybrid (DAuNC)(41.6±3.31 nm) including fine gold nanoparticles (4.5±0.9 nm) could be identified (right of FIG. 3(B)). Further, energy-dispersive X-ray spectroscopy (EDX) was performed on the ultrafine and fine gold nanoparticle cluster-protein nanoparticle hybrids, and as a result, it could be confirmed that a metal bound to the surface of the ultrafine and fine gold-protein nanoparticle hybrids was gold. (FIG. 3(C)).

### <Example 6> Establishment of Correlation Between Amount of Protein Nanoparticles of Ultrafine and Fine Gold Nanoparticle Cluster-Protein Nanoparticle Hybrids and Amount of Surface Gold Nanoparticles

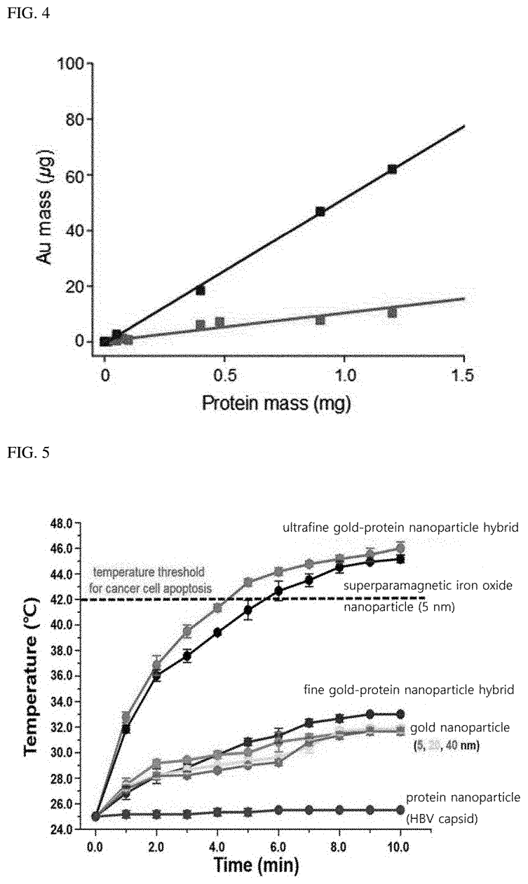

In order to establish the correlation between the amounts of the HBV capsid which is a protein nanoparticle and gold nanoparticles synthesized on its surface, in the SPAuNC and DAuNC, the amount of gold synthesized on their surface at various concentrations of protein nanoparticles was quantitatively analyzed through ICP-MS, and the correlation for this was schematically illustrated (FIG. 4). Furthermore, based on the graph results, the relationship equation between the amount of protein nanoparticles and the amount of surface gold nanoparticles for each gold-protein nanoparticle hybrid was established as follows. The relationship equation of the ultrafine gold nanoparticle cluster-protein nanoparticle hybrid is as follows in Equation 1:

$$Y ( mg ) = 0.01 X ( mg ) ( {R}^{2} = 0.95 ) \qquad [ Equation 1 ]$$

The relationship equation of the fine gold nanoparticle cluster-protein nanoparticle hybrid is as follows in Equation 2:

$$Y ( mg ) = 0.052 X ( mg ) ( {R}^{2} = 0.98 ) \qquad [ Equation 2 ]$$

(in Equations 1 and 2, Y is a mass of gold nanoparticles, and X is a mass of protein nanoparticles.)

### <Example 7> Verification of Efficacy of Magnetic Hyperthermia Through Analysis of Temperature Elevation Tendency of Ultrafine or Fine Gold Nanoparticle Cluster-Protein Nanoparticle Hybrid in Magnetic Field—In Vitro

To confirm the applicability of the prepared SPAuNC and DAuNC to magnetic hyperthermia, a temperature elevation potential in an alternating magnetic field was confirmed. Based on the above-described Equation 1 or 2, after the hybrid was mixed with 100 μl of PBS based on 2 sg of gold nanoparticles, the resulting mixture was treated with a 360 kHz alternating magnetic field with an intensity of 10 kW, and then the change in temperature over time was measured. In addition, the result was compared with the temperature elevation potentials of equal amounts of commercially available gold nanoparticles having a size of 5, 20, and 40 nm, superparamagnetic iron oxide nanoparticles known to be excellent in magnetic thermal effects, and the HBV capsid which is a protein nanoparticle. As a result, no remarkable temperature elevation aspect was seen in the DAuNC, the commercially available gold nanoparticles, and the HBV capsid protein nanoparticles. In contrast, in the case of a solution including SPAuNC, the temperature was rapidly elevated, and thus reached 44° C. within 10 minutes, thereby exhibiting a temperature elevation potential at a level similar to those of superparamagnetic iron oxide nanoparticles. (FIG. 5). This means that the temperature was elevated to a temperature equal to or more than the temperature at which cancer cells may become necrotic, confirming excellent magnetic thermal effects of the ultrafine gold nanoparticle cluster-protein nanoparticle hybrid according to the present invention.

### <Example 8> Verification of Magnetic and Physical Characteristics of Superparamagnetic Gold Nanoparticle Cluster-Protein Nanoparticle Hybrid

In order to confirm physical and magnetic characteristics of the superparamagnetic gold nanoparticle cluster-protein nanoparticle hybrid exhibiting an excellent temperature elevation potential, the measurement technique as follows was used.

1) Electron paramagnetic resonance (EPR) spectroscopy and a superconducting quantum interference device (SQUID) magnetometer analysis were performed in order to confirm magnetic characteristics of the superparamagnetic gold nanoparticle cluster-protein nanoparticle hybrid. For the analysis of each material, the ultrafine or fine gold nanoparticle cluster-protein nanoparticle hybrid was pre-freezed at −80° C. for 3 hours, and then completely lyophilized using a lyophilizer. For EPR analysis, the completely lyophilized ultrafine or fine gold nanoparticle cluster-protein nanoparticle hybrid sample was injected into an EPR spectrometer, and then magnetic characteristics of each material were analyzed.

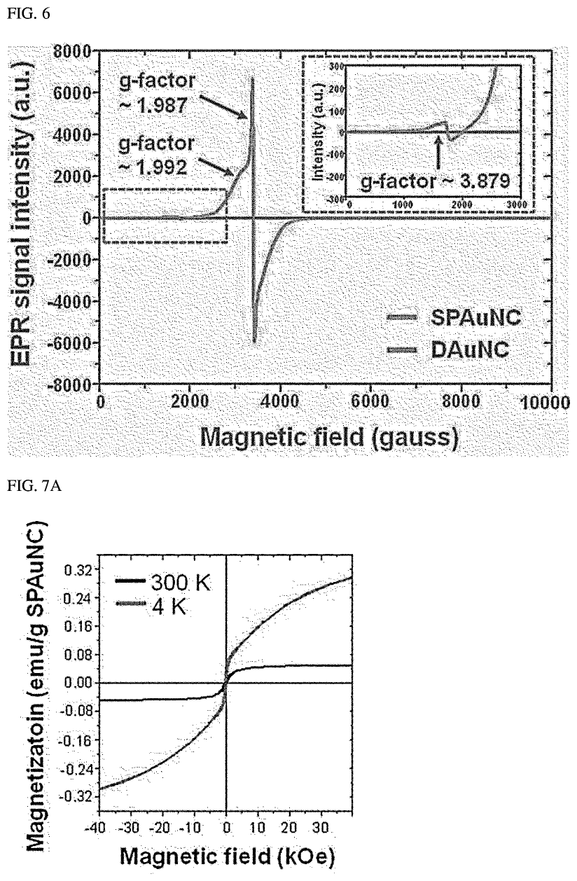

First, as a result of the EPR analysis, in the EPR spectrum of SPAuNC, EPR signals were observed at about 1700G, 3200G, and 2000 to 4500G, whereas no specific EPR signal was observed in the DAuNC (FIG. 6). Specifically, the signal at 1700G means a triplet spin state of unpaired electrons in Au atoms of SPAuNC. A sharp and strong signal at 3200G and a broad signal at 2000G to 4500G suggest the presence of separated/localized and moved/delocalized spins. Accordingly, it could be seen that the magnetism of SPAuNC was induced by the interaction between localized spins and delocalized spins in the d-band of gold atoms on the surface of SPAuNC.

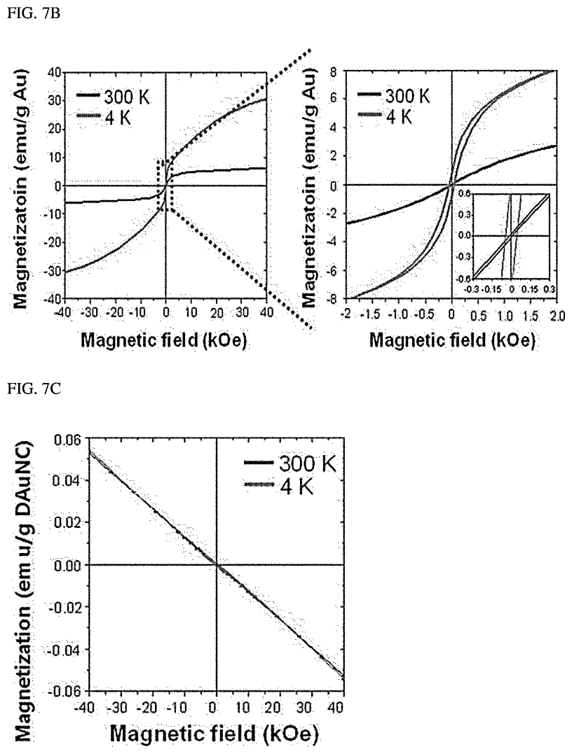

Meanwhile, in the SQUID analysis, typical superparamagnetic behavior of SPAuNC was clearly observed at 4K and 300K (FIG. 7(A)), whereas typical paramagnetic characteristics were observed in DAuNC (FIG. 7(C)). The magnetization degree of AuNPs in SPAuNC reached 4.7 emu/g Au [or 0.16μ_{B} (magnetic moment)/Au atom] at 10 kOe of 300 k (FIG. 7(B)), and this result had a value which was twice or more than that of a commercially available superparamagnetic standard [1.6 emu/g Gd ((Magnevist®, Bayer Health Care, Leverkusen, Germany) and 2.4 emu/g SPION (Resovist®, Schering AG, Berlin-Wedding, Germany)] at 10 kOe of 300 K.

2) By measuring the change in binding energy of the superparamagnetic gold nanoparticle cluster-protein nanoparticle hybrid, a gold ion source chloro(trimethyl phosphine) gold (I), and the HBV capsid protein nanoparticles using X-ray photoelectron spectroscopy (XPS), the change in binding energy of elements present on the surface of the protein nanoparticles and known to play an important role in the process or reducing gold nanoparticles was measured. Based on the change in binding energy of the corresponding element, an atom serving as a charge carrier during the process of reducing gold nanoparticles on the surface of protein nanoparticles was presumed. In order to make XPS measurement samples, aqueous solutions of the gold-protein nanoparticle hybrid, the gold ion source chloro(trimethyl phosphine) gold (I), and the HBV capsid protein nanoparticles were each drop-coated onto the surface of a silicon wafer, and then dried in a vacuum chamber. Thereafter, peaks analyzed through an analysis apparatus were fitted by XPS software, and based on this, the final change in binding energy of each element was measured.

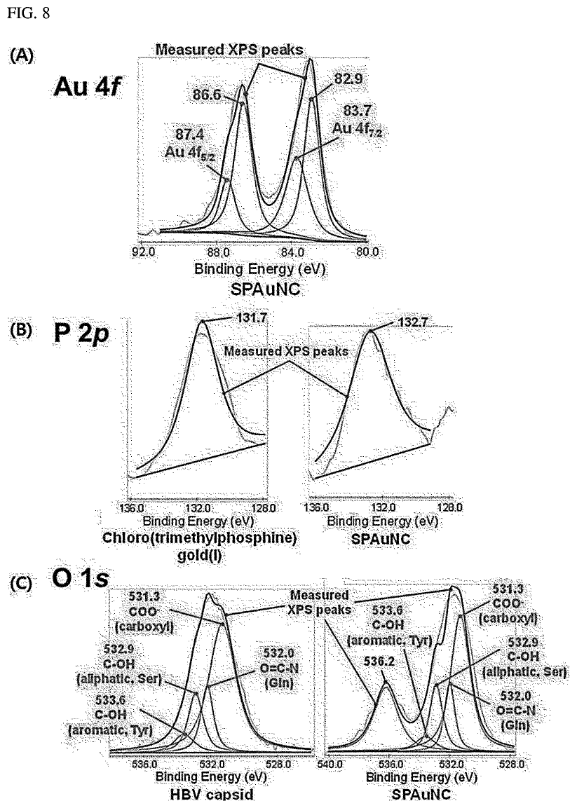

In relation to the Au binding energy of SPAuNC, peaks of 83.7 eV and 87.4 eV coincide with the peaks of bulky gold atoms (Au 4f_{7/2} and Au 4f_{5/2}, respectively), and thus, peaks of 82.9 eV and 86.6 eV exhibited the unoccupied/depleted d-band state of the surface Au atom in SPAuNC (FIG. 8, top). In addition, the difference in P binding energy of phosphine phosphorus between SPAuNC (132.7 eV) and chloro(trimethyl phosphine) gold (I)/(CH₃)₃PAuCl (131.7 eV) showed that charge moved in the direction of phosphine phosphorus on the surface of the Au atom of SPAuNC (FIG. 8, middle). Meanwhile, in SPAuNC, four peaks of O 1s were confirmed as follows: 1) aromatic C—OH (533.6 eV) of tyrosine, 2) aliphatic C—OH (532.9 eV) of serine, 3) COO— (531.3 eV), and 4) CN of O=glutamine (532.0 eV). In particular, when compared with the HBV capsid, a new O 1s peak (536.2 eV) was observed in SPAuNC, which may be presumed to be oxygen present around His₆ bound through a coordination bond with (CH₃)₃PAu⁺, that is, oxygen in the side chain of Tyr₆. That is, when compared with the O binding energy (533.6 eV) of aromatic C—OH of unreacted tyrosine, the increased binding energy (536.2 eV) of oxygen chemisorbed onto gold of Tyr₆ shows that charge moves from the surface Au atom to adjacent oxygen atoms in the side chain of Tyr₆ (bottom of FIG. 8).

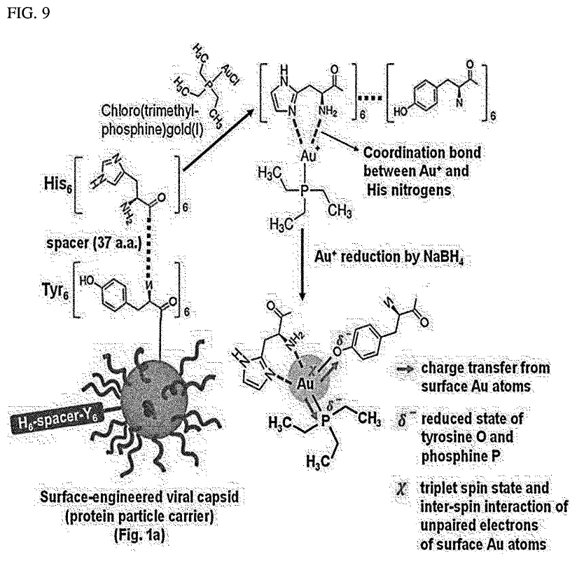

When the aforementioned results are taken together, first, the phosphine-gold ion [(CH₃)₃PAu⁺] is chemisorbed onto the engineered HBV capsid through a coordination bond between (CH₃)₃PAu⁺ and His₆. Thereafter, the chemisorbed (CH₃)₃PAu⁺ AuNPs is formed to have a size less than 2 nm under optimal reduction conditions (NaBH₄). Thereafter, ultrafine AuNP chemically binds to phosphine phosphorus, and is chemisorbed onto adjacent oxygen in the side chain of Tyr₆. Thereafter, the charge moves in a direction from the surface Au atom of AuNP to phosphine phosphorous and oxygen of tyrosine, so that an empty d state occurs to cause an interaction between localized spins and delocalized spins in the d-band of the Au surface atom. Through a series of processes, the SPAuNC of the present invention ultimately has superparamagnetic characteristics (FIG. 9).

### <Example 9> Verification of Cancer Cell Target Aiming of Prepared Superparamagnetic Gold Nanoparticle Cluster-Protein Nanoparticle Hybrid—In Vitro

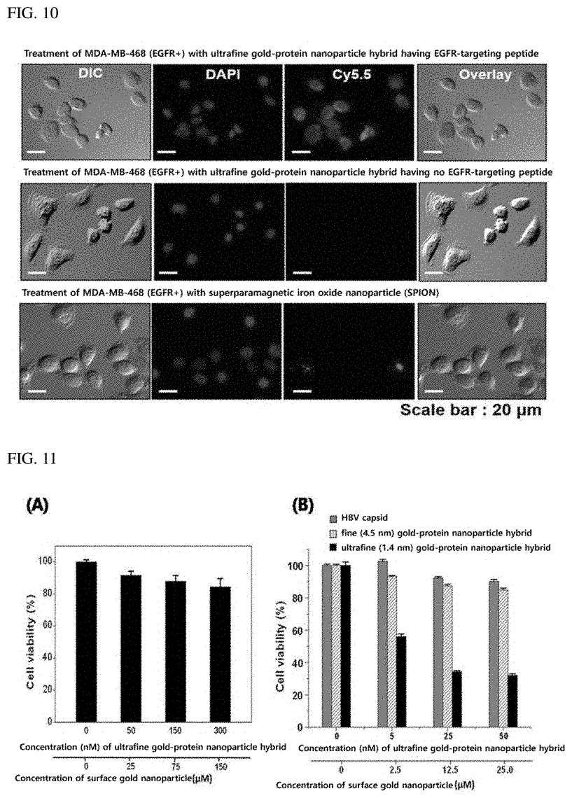

In order to verify target aiming for EGFR-overexpressing cancer cells of the superparamagnetic gold nanoparticle cluster-protein nanoparticle hybrid including the EGFR-targeting peptide, a target aiming verification experiment was performed using a human breast cancer cell line (MDA MB-468 cell line) known to overexpress EGFR. Specifically, human breast cancer cells were grown on 35 phi cell culture plates, and the N-termini of two types of superparamagnetic gold nanoparticle cluster-protein nanoparticle hybrids (a superparamagnetic gold nanoparticle cluster-protein nanoparticle hybrid having no EGFR-targeting peptide, and a superparamagnetic gold nanoparticle cluster-protein nanoparticle hybrid including an EGFR-targeting peptide) in the presence and absence of the EGFR affibody obtained in Example 1 and the surface of superparamagnetic iron oxide nanoparticles (SPION) which is a commercially available magnetic hyperthermia agent were labeled with Cy5.5 (λex=675 nm/λem=694 nm). Thereafter, a cellular particle uptake experiment was performed by treating human breast cancer cells with the material for 1 hour. As a result, fluorescence by Cy5.5 was observed only in the cell line treated with the superparamagnetic gold nanoparticle cluster-protein nanoparticle hybrid including the EGFR-targeting peptide. That is, it could be seen that the hybrid uptake of the human breast cancer cell line was remarkably improved by the specific interaction between the EGFR-targeting peptide and the EGFR peptide, and the cell targeting ability could be enhanced through the EGFR-mediated-endocytosis (FIG. 10). Further, based on the technical theory in the related art, the intracellular localization of SPAuNC could be confirmed by analyzing the fluorescent cell images: that is, a spotted fluorescence pattern means that fluorescent molecules are typically present in the endosomal compartment, whereas spread/diffused fluorescence signals exhibited signals in the cytoplasm, accordingly, it could be confirmed that the SPAuNC of the present invention was present in the cytoplasm.

### <Example 10> Verification of Cancer Cell Magnetic Hyperthermia Efficacy and Stability of Superparamagnetic Gold Nanoparticle Cluster-Protein Nanoparticle Hybrid—In Vitro

For EGFR-overexpressing breast cancer cells absorbing the superparamagnetic gold nanoparticle cluster-protein nanoparticle hybrid, it was intended to confirm 1) toxicity of the material itself affecting the cells when the cancer cells were not treated with an alternating magnetic field, or 2) effects of killing cancer cells by magnetic hyperthermia effects after the cancer cells were treated with an alternating magnetic field. In order to confirm the toxicity of the material itself before the cancer cells were treated with an alternating magnetic field, first, an MDA-MB-468 cell line, 5,000 EGFR-overexpressing breast cancer cells per well, was cultured in a 96 well plate. Thereafter, the cell line was treated with the superparamagnetic gold nanoparticle hybrid at various concentrations (0, 50, 150, and 300 sg based on protein) for 24 hours, and then the cell viability of breast cancer cells for each case was measured using a CCK-8 kit. As a result, when the cell line was not treated with the alternating magnetic field, it was confirmed that even though an excessive amount of superparamagnetic gold nanoparticle cluster-protein nanoparticle hybrid was administered, cell specific toxicity was not exhibited, so that 80% or more of the MDA-MB-468 cells survived (FIG. 11(A)).

Further, when cancer cells absorbing the ultrafine gold nanoparticle cluster-protein nanoparticle hybrid were treated with an alternating magnetic field (36 kHz, 10 kW, 30 min), excellent magnetic hyperthermia effects were exhibited as compared to HBV capsid protein nanoparticles as a control and the fine gold nanoparticle cluster-protein nanoparticle hybrid, and it could be confirmed that a large amount of cancer cells became necrotic (FIG. 11(B)). From the results, it was verified that based on a temperature elevation potential on the PBS verified in Example 7, the superparamagnetic gold nanoparticle cluster-protein nanoparticle hybrid could be used for the magnetic hyperthermia of cancer cells.

### <Example 11> Verification of Function of Superparamagnetic Gold Nanoparticle Cluster-Protein Nanoparticle Hybrid as Magnetic Resonance Imaging Contrast Agent—In Vitro

In order to confirm whether the superparamagnetic gold nanoparticle cluster-protein nanoparticle hybrid could be utilized as a magnetic resonance imaging contrast agent using the superparamagnetic property of gold nanoparticles on the surface of the superparamagnetic gold nanoparticle cluster-protein nanoparticle hybrid, the functionality as T1 and T2 contrast agents for the corresponding material was verified.

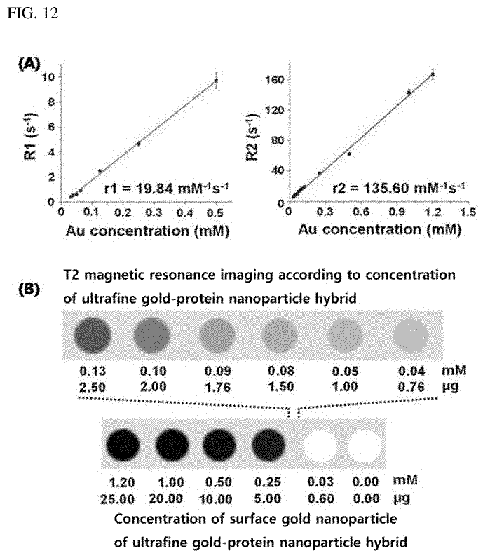

For the superparamagnetic gold nanoparticle cluster-protein nanoparticle hybrid, the T1 and T2 relaxivity was according to the concentration under the in vitro conditions was measured by utilizing an MRI apparatus having an intensity of 4.7-T. While the experiment was performed, the T1 relaxation time was measured under the following conditions: TR=100-5000 ms, TE=8.25 ms, FOV=60×40 mm, matrix=192 128, and slice thickness=1.0 mm. Meanwhile, T2 relaxation time was measured under the following conditions: TR=2000 ms, TE=6.5-208 ms, FOV=60×40 mm, matrix=192×128, and slice thickness=0.8 mm. In general, it is known that the higher the ratio of r₂ to r₁ is, the more suitable the contrast agent is for T2-weighted imaging. In the case of the superparamagnetic gold nanoparticle cluster-protein nanoparticle hybrid of the present invention, the ratio of r₂/r₁ was observed to be 6.8, which is a very high value, and comparable to the result (6.2) of a currently commercially available Resovist®, so that it was confirmed that it is highly likely for the material to be utilized as a T2 contrast agent (FIG. 12(A)). Further, it was intended to confirm the T2 MRI contrast effects of the superparamagentic gold nanoparticle cluster-protein nanoparticle hybrid according to concentration, and the present experiment was performed under the same conditions as those for measuring the T2 relaxation time. As a result, it could be confirmed that as the concentration of the superparamagnetic gold nanoparticle cluster-protein nanoparticle hybrid, that is, the concentration of the ultrafine gold nanoparticles on the surface was increased, the T2 contrast effects were increased in proportion to the increase in concentration (FIG. 12(B)).

### <Example 12> Verification of In Vivo Targeting Function of Superparamagnetic Gold Nanoparticle Cluster-Protein Nanoparticle Hybrid—In Vivo

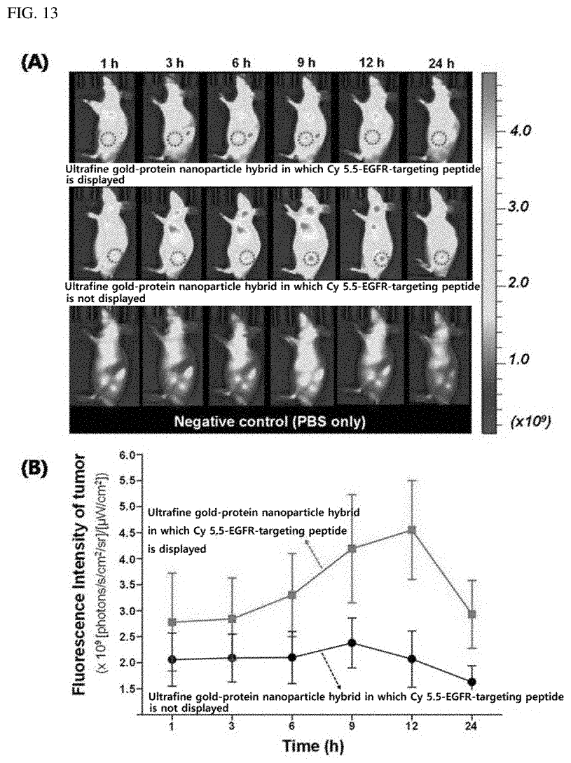

For the superparamagnetic gold nanoparticle cluster-protein nanoparticle hybrid whose efficacy had been verified at the cell level, in vivo target aiming was verified. For an animal model (BALb/c nude mouse) in which breast cancer cells (MDA-MB-468) known to overexpress EGFR were implanted subcutaneously or in the liver, the distribution of the in vivo nanoparticle hybrid was confirmed over the passage of time. For the present experiment, by subcutaneously injecting 3×10⁷ EGFR-overexpressing breast cancer cells into an animal (BALb/c nude mouse), an animal model in which EGFR-overexpressing cancer had been formed in the hypoderm was established. Thereafter, the N-termini of two types of superparamagnetic gold nanoparticle cluster-protein nanoparticle hybrids (a superparamagnetic gold nanoparticle cluster-protein nanoparticle hybrid having no EGFR-targeting peptide, and a superparamagnetic gold nanoparticle cluster-protein nanoparticle hybrid including an EGFR-targeting peptide) in the presence and absence of the EGFR affibody obtained in Example 1 were labeled with Cy5.5 (λex=675 nm/λem=694 nm), and then 0.5 μg of the ultrafine gold nanoparticle cluster-protein nanoparticles were intravenously injected based on the amount of surface gold nanoparticles. Thereafter, the in vivo distribution of each particle was measured over time by analyzing the fluorescence intensity over time through fluorescence image analysis. As a result, it was verified in vivo that the superparamagnetic gold nanoparticle cluster-protein nanoparticle hybrid according to the present invention was effectively delivered to EGFR-overexpressing cancer cells (FIGS. 13(A) and 13(B)).

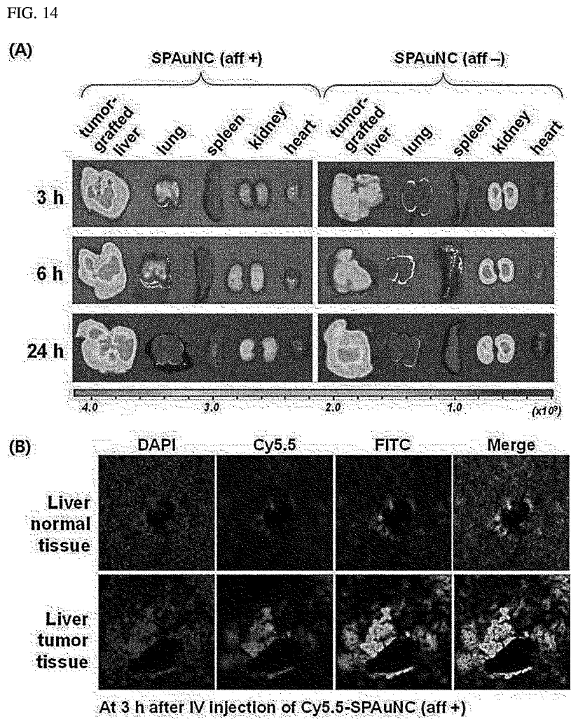

Further, for the animal model (BAL B/C nude mouse) in which EGFR-overexpressing breast cancer cells were implanted in the liver, in vivo target aiming was verified. In order to make an animal model in which EGFR-overexpressing breast cancer cells were implanted in the liver, 3×10⁷ EGFR-overexpressing breast cancer cells were injected into the left lobe of a liver of a mouse (BAL B/C nude mouse) by cutting open the abdomen of the mouse. For the corresponding manufactured animal model, the cancer cell delivery abilities of the two types of ultrafine gold nanoparticle cluster-protein nanoparticle hybrids in the presence and absence of the EGFR affibody were compared like above. As a result, it was confirmed that the superparamagnetic gold nanoparticle cluster-protein nanoparticle hybrid according to the present invention was delivered more specifically to a liver tissue into which cancer cells were implanted (FIG. 14(A)). In addition, after the excised liver was fixed in a tissue fixing solution (4 (v/v) % formaldehyde), the fixed tissue was embedded in a paraffin film, and sliced into 5-μm thicknesses. Thereafter, a change in tissue was observed by a confocal microscope by immunofluorescence staining of the tissue section. As a result, it was confirmed that the superparamagnetic gold nanoparticle cluster-protein nanoparticle hybrid according to the present invention was delivered to EGFR-overexpressing cancer cells instead of normal cells even in the liver tissue (FIG. 14(B)).

### <Example 13> Verification of In Vivo Magnetic Hyperthermia Efficacy of Superparamagnetic Gold Nanoparticle Cluster-Protein Nanoparticle Hybrid—In Vivo

In order to verify the in vivo magnetic hyperthermia effects of the superparamagnetic gold nanoparticle cluster-protein nanoparticle hybrid, first, an experiment was performed on an animal model (BALb/c nude mouse) in which a breast cancer cell line (MDA-MB-468) known to overexpress EGFR was implanted subcutaneously. For the animal model in which cancer cells were implanted subcutaneously, established in Example 12, 1) when the animal model was treated with an alternating magnetic field (360 kHz, 10 kW, 10 min) 9 hours after administering SPAuNC in an amount corresponding to 1.5 μg based on the amount of surface gold nanoparticles via intravenous injection, and 2) when only the alternating magnetic field was applied without administration of SPAuNC, the changes in size of cancer (tumor volume) were observed and compared over the passage of time. In addition, the body weight of the animal model and the size of cancer were quantified, and the size of cancer was calculated through an equation of (major axis)²×(minor axis)×0.5 (mm²).

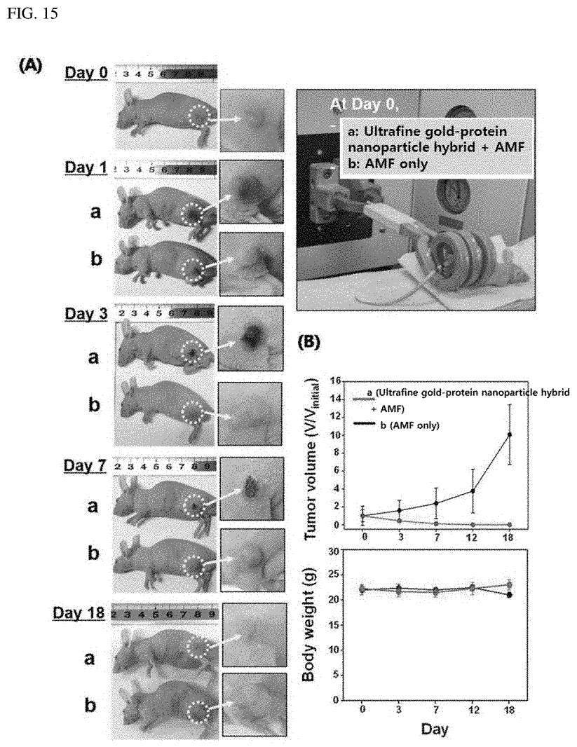

As a result, when the superparamagnetic gold nanoparticle cluster-protein nanoparticle hybrid was administered, and then the animal model was treated with an alternating magnetic field, cancer was completely treated or removed, and through this, a cancer therapeutic effect of the superparamagnetic gold nanoparticle cluster-protein nanoparticle hybrid by magnetic hyperthermia was confirmed. In contrast, when the animal model was treated with only an alternating magnetic field without administration of the superparamagnetic gold nanoparticle cluster-protein nanoparticle hybrid, the therapeutic effect was not observed at all, and it could be observed that the size of cancer continuously increased. Accordingly, the magnetic hyperthermia effect of the superparamagnetic gold nanoparticle cluster-protein nanoparticle hybrid against cancer implanted in the hypoderm was verified in vivo (FIGS. 15(A) and 15(B)).

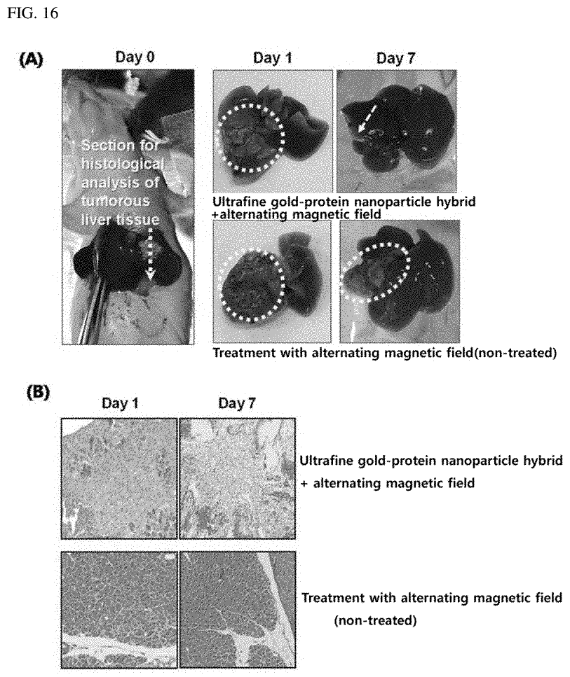

For the animal model in which cancer cells were implanted in the liver, established in Example 12, 1) an alternating magnetic field is applied 3 hours after administering the superparamagnetic gold nanoparticle cluster-protein nanoparticle hybrid in an amount corresponding to 1.5 μg based on the amount of surface gold nanoparticles via intravenous injection, and 2) when only the alternating magnetic field was applied (negative control), the magnetic hyperthermia effects of the two cases were confirmed. As a result, the cancer cell killing effect was observed only when the animal model was treated with the superparamagnetic gold nanoparticle cluster-protein nanoparticle hybrid and the alternating magnetic field (FIGS. 16(A) and 16(B)). Accordingly, the magnetic hyperthermia effect of the superparamagnetic gold nanoparticle cluster-protein nanoparticle hybrid against deep tissue cancer was verified in vivo.

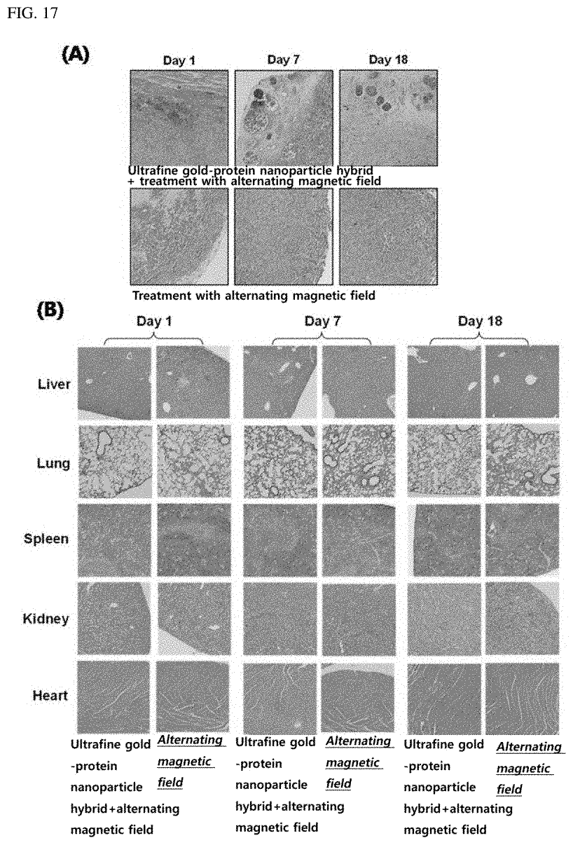

Additionally, in order to confirm effects of the cancer treatment by the magnetic hyperthermia on normal organ tissues other than cancer, H&E staining was performed on various tissues. On day 1, day 7, and day 18 after the treatment was performed by injecting the ultrafine gold-protein nanoparticles, 1) from a group into which the ultrafine gold-protein nanoparticles were injected and which was treated with the magnetic field, and 2) from a group which was treated with only the magnetic field, the heart, the liver, the lungs, the spleen, the kidney, and the pancreas were excised, and fixed in a tissue fixing solution (4 (v/v) % formaldehyde). The fixed tissue was embedded in a paraffin film, sliced into 5-μm thickness, and then stained with hematoxylin & eosin (H&E) to confirm each tissue by a microscope. As a result, it was confirmed that when the ultrafine gold-protein nanoparticles were injected and the animal model was treated with an alternating magnetic field, there was no damage to normal organ tissues as in the group which was treated with only the magnetic field (FIG. 17).

### <Example 14> Verification of Efficacy of Superparamagnetic Gold Nanoparticle Cluster-Protein Nanoparticle Hybrid as In Vivo Magnetic Resonance Imaging Contrast Agent—In Vivo

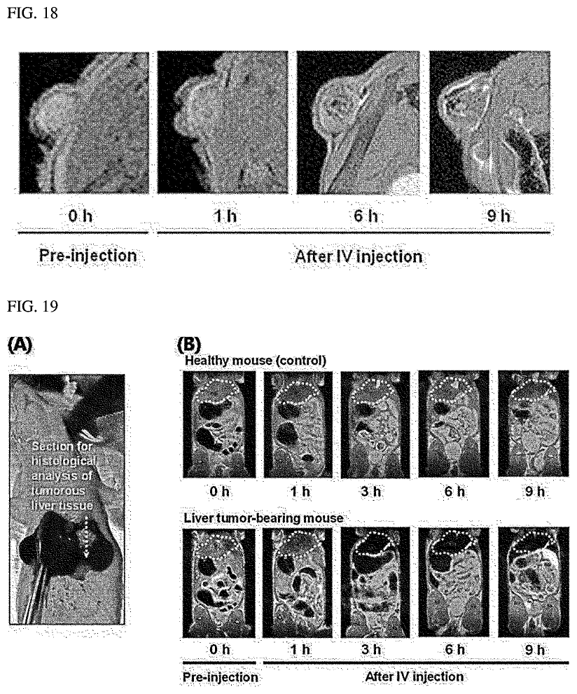

In order to verify the efficacy of the efficacy of the superparamagnetic gold nanoparticle cluster-protein nanoparticle hybrid as a contrast agent of in vivo magnetic resonance imaging, an experiment was performed on the animal model (BALb/c nude mouse) in which an EGFR-overexpressing breast cancer cell line (MDA-MB-468) was implanted subcutaneously, established in Example 12. After the superparamagnetic gold nanoparticle cluster-protein nanoparticle hybrid was administered via intravenous injection in an amount corresponding to 1.5 μg based on the amount of surface gold nanoparticles to the animal model in which cancer cells were implanted subcutaneously. T2 MRI imaging was performed using a magnetic resonance imaging apparatus having an alternating magnetic field intensity of 4.7-T. In this case, the mice were anesthetized by breathing a mixed gas of oxygen and 1% isofluorane, and an experiment was performed on an animal-dedicated bed to maintain body temperature. T2 magnetic resonance imaging was performed under the following conditions: TR=3500 ms, TE=32.118 ms, FOV=35×35 mm, matrix=256×256, and slice thickness=1.0 mm, NEX=2, flip angle=180° C. As a result of performing the magnetic resonance imaging on the animal model in which cancer was implanted subcutaneously, an EGFR-overexpressing cancer implanted site in the hypoderm was clearly contrasted over time, and thus appeared darker than sites around the implanted site, and based on this, the efficacy of the superparamagnetic gold nanoparticle cluster-protein hybrid as a T2-MRI contrast agent was verified in vivo (FIG. 18).

After the superparamagnetic gold nanoparticle cluster-protein nanoparticle hybrid in an amount corresponding to 1.5 μg and PBS were respectively intravenously injected into the deep tissue cancer animal model (BAL B/C nude mouse) manufactured by implanting the EGFR-overexpressing breast cancer cell line (MDA-MB-468) into the liver established in Example 13 and normal mice as a control, T2 MRI imaging was performed using a magnetic resonance imaging apparatus having an alternating magnetic field intensity of 4.7-T. In this case, the mice were anesthetized by breathing a mixed gas of oxygen and 1% isofluorane, and an experiment was performed on an animal-dedicated bed to maintain body temperature. T2 magnetic resonance imaging was performed under the following conditions: TR=3500 ms, TE=32.118 ms, FOV=35×35 mm, matrix=256×256, and slice thickness=1.0 mm, NEX=2, flip angle=180° C. As a result of comparing the magnetic resonance imaging of the control normal mice and the deep tissue cancer animal model over the passage of time, the T2 contrast effect of the cancer site could be clearly confirmed in only the deep tissue cancer animal model (FIG. 19). Accordingly, it was confirmed that in magnetic resonance imaging-based diagnosis against subcutaneous cancer and deep tissue cancer, the superparamagnetic gold nanoparticle cluster-protein nanoparticle hybrid according to the present invention could be utilized as a T2-MRI contrast agent.

### <Example 15> Confirmation of Whether Surface Gold Nanoparticles of Superparamagnetic Gold Nanoparticle Cluster-Protein Nanoparticle Hybrid are Accumulated In Vivo—In Vivo

After 40 nm gold nanoparticles or the superparamagnetic gold nanoparticle cluster-protein nanoparticle hybrid in an amount of 300 μg based on the mass of gold nanoparticles were administered via intravenous injection into each normal animal (C57BL/6 mouse) in which cancer was not implanted, the residual amount of gold accumulated in each organ over time was analyzed through ICP-MS. On days 1, 4, 7, 10, 14, 21, 28, 35, and 42 after injection, the liver, the pancreas, the kidneys, the heart, and the femur were excised from animals of each group, fixed in a tissue fixing solution (4 (v/v) % formaldehyde), and then dried at 60° C. overnight. Thereafter, after the dried organs were stored in a 30% aqueous hydrogen peroxide solution for 1 day, gold in the organ was completely dissolved using aqua regia in which nitric acid and sulfuric acid were mixed at a ratio of 1:3. Thereafter, each sample was diluted 1/10 with hydrochloric acid, and filtered by a 0.45 mm Teflon filter. Finally, the amount of gold in the filtered solution was quantitatively analyzed using ICP-MS.

As a result, as shown in the following Table 2, it was confirmed that in the case of the superparamagnetic gold nanoparticle cluster-protein nanoparticle hybrid, about 21% of the amount of gold initially administered was accumulated in one day, but the amount was reduced to less than 1% in 6 weeks. It was observed that in the case of gold nanoparticles (40 nm), about 38% of the amount of gold initially administered was accumulated in the liver in only one day, and even after 6 weeks, about 19% continuously remained in the liver (a significant amount of gold nanoparticles were not detected in the heart and the femur). That is, in the case of the superparamagnetic gold nanoparticle cluster-protein nanoparticle hybrid, the residual amount of gold in the liver was rapidly decreased, and simultaneously, a large amount of gold was detected in the kidneys, from which it could be inferred that gold nanoparticles accumulated in the liver passed through the kidneys and were released ex vivo through urine.

TABLE 2
Au contents μg (Au) of % of the given dose¹

| | Liver | | Spleen | | Kidney | |
|---|---|---|---|---|---|---|
| | [symbol] | | [symbol] | | [symbol] | |
| | [symbol] | | [symbol] | | [symbol] | |
| | [symbol] | Synthetic | [symbol] | Synthetic | [symbol] | Synthetic |
| Time | [symbol] | AuNP | [symbol] | AuNP | [symbol] | AuNP |
| (day) | [symbol] | (40 nm) | [symbol] | (40 nm) | [symbol] | (40 nm) |
| 1 | 63.9/21.3 | 112.6/37.5 | 0.6/0.2 | 0.5/0.02 | 6.4/2.1 | ND² |
| 4 | 39.2/13.1 | 72.9/24.3 | 0.5/0.2 | 0.5/0.2 | 6.9/2.3 | ND |
| 7 | 29.2/9.7 | 66.3/22.1 | 0.4/0.1 | 0.6/0.2 | 6.0/2.0 | 0.4/0.1 |
| 10 | 24.5/8.2 | 61.7/20.6 | 0.3/0.1 | 0.6/0.2 | 4.0/1.3 | 0.3/0.1 |
| 14 | 21.1/7.0 | 60.5/20.2 | 0.2/0.1 | 0.6/0.2 | 2.7/0.9 | 0.2/0.1 |
| 21 | 12.9/4.3 | 59.7/19.9 | 0.1/0.03 | 0.5/0.2 | 2.2/0.7 | 0.1/0.03 |
| 28 | 8.4/2.8 | 58.2/19.4 | 0.1/0.03 | 0.3/0.1 | 1.4/0.5 | ND |
| 35 | 3.6/1.2 | 58.2/19.4 | 0.1/0.03 | 0.2/0.1 | 0.5/0.2 | ND |
| 42 | 2.6/0.9 | 55.9/18.6 | 0.1/0.03 | 0.2/0.1 | 0.3/0.01 | ND |

¹Amount of initial dose is 300.0 μg Au (12.0 μg Au/g mouse)
²Not detected (limit of detection Au in ICP 20 ppb) [symbol]
indicates data missing or illegible when filed

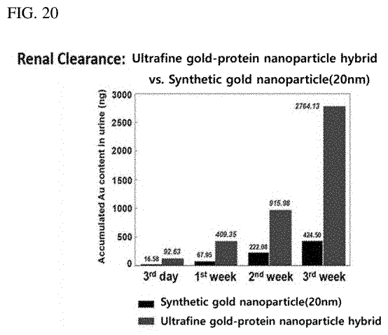

In order to more specifically confirm the aforementioned experimental results, 42.6 μg of 20 nm gold nanoparticles and the superparamagnetic gold nanoparticle cluster-protein nanoparticle hybrid in an amount corresponding to 42.6 μg based on the amount of gold nanoparticles were intravenously injected, and urine was collected from mice of each group every 30 minutes for 6 hours at the time points when 3 days, 7 days, 14 days, and 21 days elapsed. After the collected urine was classified according to the day/group, the total volume was diluted to 1 mL. Thereafter, after the diluted urine was stored in a 30% aqueous hydrogen peroxide solution for 1 day, gold in the urine was completely dissolved using an aqueous regia in which nitric acid and sulfuric acid were mixed at a ratio of 1:3. Thereafter, each sample was diluted 1/10 with hydrochloric acid, and filtered by a 0.45 mm Teflon filter. Finally, the amount of gold in the filtered solution was quantitatively analyzed using ICP-MS. As a result, it was confirmed that compared to 20 nm gold nanoparticles, the superparamagnetic gold nanoparticle cluster-protein nanoparticle hybrid was released ex vivo in a short time, which coincided with the analysis result of the residual amount of gold in the excised organs. Therefore, the aforementioned experimental results show that the possibility of causing toxicity/side effects due to the in vivo accumulation of the superparamagnetic gold nanoparticle cluster-protein nanoparticle hybrid is very low (FIG. 20).

### <Example 16> Confirmation of In Vivo Stability of Surface Gold Nanoparticles of Superparamagnetic Gold Nanoparticle Cluster-Protein Nanoparticle Hybrid—In Vivo

After the superparamagnetic gold nanoparticle cluster-protein nanoparticle hybrid was administered in an amount of 300 μg based on the mass of gold nanoparticles via intravenous injection into normal animals (C57BL/6 mouse) in which cancer was not implanted, the residual amount of gold in blood over time was analyzed through ICP-MS. At 1, 3, 6, 12, and 24 hours after administration, blood was collected from the animals of each group. After the blood was stored in a 30% aqueous hydrogen peroxide solution for 1 day, gold in blood was completely dissolved using aqua regia in which nitric acid and sulfuric acid were mixed at a ratio of 1:3. Thereafter, each sample was diluted 1/10 with hydrochloric acid, and filtered by a 0.45 mm Teflon filter. Finally, the amount of gold in the filtered solution was quantitatively analyzed using ICP-MS.

As a result, as shown in the following Table 3, in the case of the superparamagnetic gold nanoparticle cluster-protein nanoparticle hybrid, the concentration of gold in blood was measured to be highest 3 hours after the injection, and after 12 hours, the concentration in blood was maintained up to almost the half level of the initial concentration after 1 hour, but after 24 hours, the amount of gold in blood was measured to be less than 20 ppb. Therefore, it could be confirmed that after the superparamagnetic gold nanoparticle cluster-protein nanoparticle hybrid was administered in vivo, the concentration was relatively stable maintained up to the time point when 12 hours elapsed.

TABLE 3

| Time | Au concentration in |
|---|---|
| (h) | blood¹ (ppb) |
| 1 | 4338.3 ± 378.4 |
| 3 | 5040.4 ± 132.4 |
| 6 | 4266.8 ± 129.7 |
| 12 | 2368.6 ± 369.6 |
| 24 | N.D.² |

¹Amount of ititial dose is 300 μg Au (12 μg Au/g mouse)
²Not detected (linit of detection of Au in ICP 20 ppb)

The above-described description of the present invention is provided for illustrative purposes, and the person skilled in the art to which the present invention pertains will understand that the present invention can be easily modified into other specific forms without changing the technical spirit or essential features of the present invention. Therefore, it should be understood that the above-described Examples are only exemplary in all aspects and are not restrictive.

The superparamagnetic gold nanoparticle cluster-protein nanoparticle hybrid according to the present invention can be applied to the fields of magnetic hyperthermia and magnetic resonance imaging contrast.

What is claimed is:

1. A method for treating cancer comprising:
administering a superparamagnetic gold nanoparticle cluster-protein nanoparticle hybrid comprising a recombinant hepatitis B virus (HBV) capsid protein nanoparticle and a superparamagnetic gold nanoparticle cluster formed on the protein nanoparticle to a subject in need of treating cancer.

2. The method according to claim 1, wherein the recombinant hepatitis B virus (HBV) capsid protein nanoparticle comprise a gold ion adsorbable peptide comprising amino acid sequences comprising a plurality of histidines (Hn, n≥2).

3. The method according to claim 2, wherein the gold ion adsorbable peptide is introduced at a N-terminus of the recombinant HBV capsid protein nanoparticle.

4. The method according to claim 1, wherein the recombinant hepatitis B virus (HBV) capsid protein nanoparticle further comprises a superparamagnetic inducing peptide comprising any one or more selected from the group consisting of a plurality of tyrosines (Yₙ, n≥2), threonines (Tₙ, n≥2), serines (Sₙ, n≥2), and cysteines (Cₙ, n≥2).

5. The method according to claim 1, wherein the recombinant hepatitis B virus (HBV) capsid protein nanoparticle further comprises a target-oriented peptide against cancer cells.

6. The method according to claim 5, wherein the target-oriented peptide is introduced at the spike site of the recombinant HBV capsid protein nanoparticle.

7. The method according to claim 6, wherein the target-oriented peptide is located between 1-78 amino acid positions and 81-149 amino acid positions, of the recombinant HBV capsid protein nanoparticle.

8. The method according to claim 5, wherein the target-oriented peptide against cancer cells targets any one selected from the group consisting of integrin, vimentin, human fibronectin extradomain B (EDB), interleukin-4 (IL-4), human epidermal growth factor receptor 2(HER-2), CD20, poliovirus receptor(PVR), VISTA, 4-1BBL, Galectin-9, Adenosine A2a receptor, CD80, CD86, ICOS, ICOSL, BTLA, OX-40L, CD155, BCL2, MYC, PP2A, BRD1, BRD2, BRD3, BRD4, BRDT, CBP, E2F1, MDM2, MDMX, PPP2CA, PPM1D, STAT3, IDH1, PD1, CTLA4, PD-L1, PD-L2, LAG3, TIM3, TIGIT, BTLA, SLAMF7, 4-1BB, OX-40, ICOS, GITR, ICAM-1, BAFFR, HVEM, LFA-1, LIGHT, NKG2C, SLAMF7, NKp80, LAIR1, 2B4, CD2, CD3, CD16, CD20, CD27, CD28, CD40L, CD48, CD52, EGFR family, AXL, CSF1R, DDR1, DDR2, EPH receptor family, FGFR family, VEGFR family, IGF1R, LTK, PDGFR family, RET, KIT, KRAS, NTRK1 and NTRK2.

9. The method according to claim 1, wherein the recombinant hepatitis B virus (HBV) capsid protein nanoparticle further comprises a linker peptide between a gold ion adsorbable peptide and a superparamagnetic inducing peptide.

10. The method according to claim 1, further comprising:
applying a magnetic field to the superparamagnetic gold nanoparticle cluster-protein nanoparticle hybrid to generate heat from the superparamagnetic gold nanoparticle cluster-protein nanoparticle hybrid.

11. A method for magnetic resonance imaging (MRI) comprising:
administering a superparamagnetic gold nanoparticle cluster-protein nanoparticle hybrid comprising a recombinant hepatitis B virus (HBV) capsid protein nanoparticle and a superparamagnetic gold nanoparticle cluster formed on the protein nanoparticle to a subject.

12. The method according to claim 11, wherein the recombinant hepatitis B virus (HBV) capsid protein nanoparticle comprises a gold ion adsorbable peptide comprising amino acid sequences comprising a plurality of histidines (Hn, n≥2).

13. The method according to claim 12, wherein the gold ion adsorbable peptide is introduced at a N-terminus of the recombinant HBV capsid protein.

14. The method according to claim 11, wherein the recombinant hepatitis B virus (HBV) capsid protein nanoparticle further comprises a superparamagnetic inducing peptide comprising any one or more selected from the group consisting of a plurality of tyrosines (Yₙ, n≥2), threonines (Tₙ, n≥2), serines (Sₙ, n≥2), and cysteines (Cₙ, n≥2).

15. The method according to claim 11, wherein the recombinant hepatitis B virus (HBV) capsid protein nanoparticle further comprises a target-oriented peptide against cancer cells.

16. The method according to claim 15, wherein the target-oriented peptide is introduced at the spike site of the recombinant HBV capsid protein nanoparticle.

17. The method according to claim 16, wherein the target-oriented peptide is located between 1-78 amino acid positions and 81-149 amino acid positions, of the recombinant HBV capsid protein nanoparticle.

18. The method according to claim 15, wherein the target-oriented peptide against cancer cells targets any one selected from the group consisting of integrin, vimentin, human fibronectin extradomain B (EDB), interleukin-4 (IL-4), human epidermal growth factor receptor 2(HER-2), CD20, poliovirus receptor(PVR), VISTA, 4-1BBL, Galectin-9, Adenosine A2a receptor, CD80, CD86, ICOS, ICOSL, BTLA, OX-40L, CD155, BCL2, MYC, PP2A, BRD1, BRD2, BRD3, BRD4, BRDT, CBP, E2F1, MDM2, MDMX, PPP2CA, PPM1D, STAT3, IDH1, PD1, CTLA4, PD-L1, PD-L2, LAG3, TIM3, TIGIT, BTLA, SLAMF7, 4-1BB, OX-40, ICOS, GITR, ICAM-1, BAFFR, HVEM, LFA-1, LIGHT, NKG2C, SLAMF7, NKp80, LA1R1, 2B4, CD2, CD3, CD16, CD20, CD27, CD28, CD40L, CD48, CD52, EGFR family, AXL, CSF1R, DDR1, DDR2, EPH receptor family, FGFR family, VEGFR family, IGF1R, LTK, PDGFR family, RET, KIT, KRAS, NTRK1 and NTRK2.

19. The method according to claim 11, wherein the recombinant hepatitis B virus (HBV) capsid protein nanoparticle further comprises a linker peptide between a gold ion adsorbable peptide and a superparamagnetic inducing peptide.

20. The method according to claim 11, wherein the MRI is performed by using a MRI apparatus having an alternating magnetic field intensity of 4.7-T.


SEQUENCE LISTING (16 sequences)

SEQ ID NO: 1 - DNA, 552 bp, Hepatitis B virus
atggacattgacccgtataaagaatttggagcttctgtggagttactctcttttttgccttctgacttctttccttctattcgagatctcctcgacaccgcctctgctctgtatcgggaggccttagagtctccggaacattgttcacctcaccatacagcactcaggcaagctattctgtgttggggtgagttgatgaatctggccacctgggtgggaagtaatttggaagacccagcatccagggaattagtagtcagctatgtcaacgttaatatgggcctaaaaatcagacaactattgtggtttcacatttcctgtcttacttttggaagagaaactgttcttgagtatttggtgtcttttggagtgtggattcgcactcctcccgcttacagaccaccaaatgcccctatcttatcaacacttccggaaactactgttgttagacgccgaggcaggtcccctagaagaagaactccctcgcctcgcagacgaaggtctcaatcaccgcgtcgcagaagatctcaatctcgggaatctcaatgttag

SEQ ID NO: 2 - DNA, 423 bp, Artificial Sequence
  N-NdeI-H6-LP-Y6-HBVcAg(1-78)-XhoI-C
catatgcatcaccatcaccatcacatggcgtctagtctgcgtcagattctggattctcagaaaatggaatggcgttctaatgcgggtggctctggtggcggaagtgggggaggcactggaggtggcagcggcggtgggtactattactattactatgacattgacccgtataaagaatttggagcttctgtggagttactctcttttttgccttctgacttctttccttctattcgagatctcctcgacaccgcctctgctctgtatcgggaggccttagagtctccggaacattgttcacctcaccatacagcactcaggcaagctattctgtgttggggtgagttgatgaatctggccacctgggtgggaagtaatttggaagacggtggcggagggagtggggggggcggtactctcgag

SEQ ID NO: 3 - DNA, 249 bp, Artificial Sequence
  N-BamHI-HBVcAg(81-149)-ClaI -C
ggatccggtggcggagggtctgggggaggcggttccagggaattagtagtcagctatgtcaacgttaatatgggcctaaaaatcagacaactattgtggtttcacatttcctgtcttacttttggaagagaaactgttcttgagtatttggtgtcttttggagtgtggatacgcactcctcccgcttacagaccaccaaatgcccctatcttatcaacacttccggaaactactgttgtttaaatcgat

SEQ ID NO: 4 - DNA, 366 bp, Artificial Sequence
  5′-XhoI-EGFR affibody-BamHI-3′
ctcgaggtggataacaaatttaacaaagaaatgtgggcggcgtgggaagaaattcgtaacctgccgaacctgaacggctggcagatgaccgcgtttattgcgagcctggtggatgatccgagccagagcgcgaacctgctggcggaagcgaaaaaactgaacgatgcgcaggcgccgaaagaattcgtggataacaaatttaacaaagaaatgtgggcggcgtgggaagaaattcgtaacctgccgaacctgaacggctggcagatgaccgcgtttattgcgagcctggtggatgatccgagccagagcgcgaacctgctggcggaagcgaaaaaactgaacgatgcgcaggcgccgaaaggatcc

SEQ ID NO: 5 - DNA, 1026 bp, Artificial Sequence
  N-H6-LP-Y6-HBVcAg(1-78)-EGFR affibody-HBVcAg(81-149)-C
catatgcatcaccatcaccatcacatggcgtctagtctgcgtcagattctggattctcagaaaatggaatggcgttctaatgcgggtggctctggtggcggaagtgggggaggcactggaggtggcagcggcggtgggtactattactattactatgacattgacccgtataaagaatttggagcttctgtggagttactctcttttttgccttctgacttctttccttctattcgagatctcctcgacaccgcctctgctctgtatcgggaggccttagagtctccggaacattgttcacctcaccatacagcactcaggcaagctattctgtgttggggtgagttgatgaatctggccacctgggtgggaagtaatttggaagacggtggcggagggagtggggggggcggtactctcgaggtggataacaaatttaacaaagaaatgtgggcggcgtgggaagaaattcgtaacctgccgaacctgaacggctggcagatgaccgcgtttattgcgagcctggtggatgatccgagccagagcgcgaacctgctggcggaagcgaaaaaactgaacgatgcgcaggcgccgaaagaattcgtggataacaaatttaacaaagaaatgtgggcggcgtgggaagaaattcgtaacctgccgaacctgaacggctggcagatgaccgcgtttattgcgagcctggtggatgatccgagccagagcgcgaacctgctggcggaagcgaaaaaactgaacgatgcgcaggcgccgaaaggatccggtggcggagggtctgggggaggcggttccagggaattagtagtcagctatgtcaacgttaatatgggcctaaaaatcagacaactattgtggtttcacatttcctgtcttacttttggaagagaaactgttcttgagtatttggtgtcttttggagtgtggatacgcactcctcccgcttacagaccaccaaatgcccctatcttatcaacacttccggaaactactgttgtttaaatcgat

SEQ ID NO: 6 - DNA, 42 bp, Artificial Sequence
  primer 1
catatgcatcaccatcaccatcacatggcgtctagtctgcgt

SEQ ID NO: 7 - DNA, 50 bp, Artificial Sequence
  primer 2
atggcgtctagtctgcgtcagattctggattctcagaaaatggaatggcg

SEQ ID NO: 8 - DNA, 51 bp, Artificial Sequence
  primer 3
cagaaaatggaatggcgttctaatgcgggtggctctggtggcggaagtggg

SEQ ID NO: 9 - DNA, 51 bp, Artificial Sequence
  primer 4
ggtggcggaagtgggggaggcactggaggtggcggcggtgggtactattac

SEQ ID NO: 10 - DNA, 48 bp, Artificial Sequence
  primer 5
ggcggtgggtactattactattactatgacattgacccgtataaagaa

SEQ ID NO: 11 - DNA, 26 bp, Artificial Sequence
  primer 6
ctcgaggtcttccaaattacttccca

SEQ ID NO: 12 - DNA, 27 bp, Artificial Sequence
  primer 7
ggatcctccagggaattagtagtcagc

SEQ ID NO: 13 - DNA, 33 bp, Artificial Sequence
  primer 8
atcgatttaaacaacagtagtttccggaagtgt

SEQ ID NO: 14 - DNA, 27 bp, Artificial Sequence
  primer 9
ctcgaggtggataacaaatttaacaaa

SEQ ID NO: 15 - DNA, 42 bp, Artificial Sequence
  primer 10
ggatcctttcggcgcctgcgcatcgttcagttttttcgcttc

SEQ ID NO: 16 - protein, 8 aa, Artificial Sequence
  FLAG tag
DYKDDDDK